(19) United States
(12) Patent Application Publication
(10) Pub. No.: US 2023/0132256 A1
(43) Pub. Date: Apr. 27, 2023

(54) COMBINATION THERAPY

(71) Applicants: ADC THERAPEUTICS SA, Epalinges (CH); MEDIMMUNE LIMITED, Cambridge (GB)

(72) Inventors: Patricius Hendrikus Cornelis VAN BERKEL, London (GB); Francesca ZAMMARCHI, London (GB); John HARTLEY, London (GB)

(21) Appl. No.: 17/259,466

(22) PCT Filed: Jul. 8, 2019
(86) PCT No.: PCT/EP2019/068287

§ 371 Date: Jan. 11, 2021

(30) Foreign Application Priority Data
Jul. 11, 2018 (GB) 1811364.7

(51) Int. Cl. A61K 47/68; A61K 31/5517; A61K 31/7068; A61P 35/00; A61K 45/06

(52) U.S. Cl. CPC A61K 47/6849; A61K 31/5517; A61K 31/7068; A61K 47/6803; A61K 47/6851; A61P 35/00; A61K 45/06

(57) ABSTRACT

The present disclosure relates to combination therapies for the treatment of pathological conditions, such as cancer. In particular, the present disclosure relates to combination therapies comprising treatment with an anti-CD25 ADC and Gemcitabine.

### EARLIER APPLICATION

This application claims priority from United Kingdom application GB1811364.7, file on 11 Jul. 2018.

### FIELD

The present disclosure relates to combination therapies for the treatment of pathological conditions, such as cancer. In particular, the present disclosure relates to combination therapies comprising treatment with an Antibody Drug Conjugate (ADC) and Gemcitabine or 5-fluorouracil.

### BACKGROUND

Antibody Therapy

Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

CD25

The type I transmembrane protein CD25 is present on activated T- and B-cells, some thymocytes, myeloid precursors, and oligodendrocytes. On activated T-cells, it forms heterodimers with the beta- and gamma subunits (CD122 and CD132), thus comprising the high-affinity receptor for IL-2. This ligand represents a survival factor for activated T-cells, as removal of IL-2 leads to immediate death of these cells.

In case of B-cells, CD25 is physiologically expressed in early developmental stages of late pro-B and pre-B cells. Malignancies arising from this stage of B-cell differentiation may thus also express CD25. Mast cell lesions are also positive for CD25 which is thus considered as a key diagnostic criterion for determination of systemic mastocytosis. In Hodgkin lymphomas, CD25 is reported to be not expressed in Hodgkin-/Reed-Sternberg cells in nodular lymphocyte predominance Hodgkin lymphoma (NLPHL), whereas the same cell type expresses CD25 at varying levels in classical Hodgkin' lymphomas of mixed cellularity type. The general expression levels are reported to be lower than in tumor infiltrating lymphocytes (TILs), which may result in problems demonstrating CD25 tumor cells in these cases (Levi et al., Merz et al, 1995).

Expression of the target antigen has also been reported for several B- and T-cell-derived subtypes of non-Hodgkin-lymphomas, i.e. B-cell chronic lymphatic leukemia, hairy cell leukemia, small cell lymphocytic lymphoma/chronic lymphocytic leukemia as well as adult T-cell leukemia/lymphoma and anaplastic large cell lymphoma.

CD25 may be localised to the membrane, with some expression observed in the cytoplasm. Soluble CD25 may also be observed outside of cells, such as in serum.

Therapeutic Uses of Anti-CD25 ADCs

The efficacy of an Antibody Drug Conjugate comprising an anti-CD25 antibody (an anti-CD25-ADC) in the treatment of, for example, cancer has been established—see, for example, WO2014/057119, WO2016/083468, and WO2016/166341.

Research continues to further improve the efficacy, tolerability, and clinical utility of anti-CD25 ADCs. To this end, the present authors have identified clinically advantageous combination therapies in which an anti-CD25 ADC is administered in combination with Gemcitabine or 5-fluorouracil.

### SUMMARY

The present authors have determined that the administration of a combination of an ADC and Gemcitabine or 5-fluorouracil to an individual leads to unexpected clinical advantages.

Accordingly, in one aspect the disclosure provides a method for treating a disorder in an individual, the method comprising administering to the individual an effective amount of an ADC and Gemcitabine or 5-fluorouracil.

The disorder may be a proliferative disease, for example a cancer such as Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be an anti-CD25-ADC, such as ADCX25 described herein.

The ADC may be anti-CD25-ADC, such as ADCT-301.

The individual may be human. The individual may have cancer, or may have been determined to have cancer. The individual may have, or have been determined to have, a CD25+ cancer or CD25+ tumour-associated non-tumour cells, such as CD25+ infiltrating T-cells.

The individual may have, or have been determined to have, a PD-L1+ cancer.

In the disclosed methods the ADC may be administered before the Gemcitabine or 5-fluorouracil, simultaneous with the Gemcitabine or 5-fluorouracil, or after the Gemcitabine or 5-fluorouracil. The disclosed methods may comprise administering a further chemotherapeutic agent to the individual.

In another aspect, the disclosure provides a first composition comprising an ADC for use in a method of treating a disorder in an individual, wherein the treatment comprises administration of the first composition in combination with a second composition comprising Gemcitabine or 5-fluorouracil.

Also provided by this aspect is a first composition comprising Gemcitabine or 5-fluorouracil for use in a method of treating a disorder in an individual, wherein the treatment comprises administration of the first composition in combination with a second composition comprising an ADC.

The disorder may be a proliferative disease, for example a cancer such as Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL), and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD25-ADC, such as ADCX25 described herein.

The ADC may be anti-CD25-ADC, such as ADCT-301.

The individual may be human. The individual may have cancer, or may have been determined to have cancer. The individual may have, or have been determined to have, a CD25+ cancer or CD25+ tumour-associated non-tumour cells, such as CD25+ infiltrating T-cells.

The individual may have, or have been determined to have, a PD-L1+ cancer.

The first composition may be administered before the second composition, simultaneous with the second composition, or after the second composition. The treatment may comprise administering a further chemotherapeutic agent to the individual.

In a further aspect, the disclosure provides the use of n ADC in the manufacture of a medicament for treating a disorder in an individual, wherein the medicament comprises an ADC, and wherein the treatment comprises administration of the medicament in combination with a composition comprising Gemcitabine or 5-fluorouracil.

Also provided by this aspect is the use of Gemcitabine or 5-fluorouracil in the manufacture of a medicament for treating a disorder in an individual, wherein the medicament comprises Gemcitabine or 5-fluorouracil, and wherein the treatment comprises administration of the medicament in combination with a composition comprising an ADC.

The disorder may be a proliferative disease, for example a cancer such as Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25−ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD25-ADC, such as ADCX25 described herein.

The ADC may be anti-CD25-ADC, such as ADCT-301.

The individual may be human. The individual may have cancer, or may have been determined to have cancer. The individual may have, or have been determined to have, a CD25+ cancer or CD25+ tumour-associated non-tumour cells, such as CD25+ infiltrating T-cells.

The individual may have, or have been determined to have, a PD-L1+ cancer.

The medicament may be administered before the composition, simultaneous with the composition, or after the composition. The treatment may comprise administering a further chemotherapeutic agent to the individual.

Another aspect of the disclosure provides a kit comprising:
a first medicament comprising an ADC;
a second medicament comprising Gemcitabine or 5-fluorouracil; and, optionally,
a package insert comprising instructions for administration of the first medicament to an individual in combination with the second medicament for the treatment of a disorder.

Also provided by this aspect is a kit comprising a medicament comprising an ADC and a package insert comprising instructions for administration of the medicament to an individual in combination with a composition comprising Gemcitabine or 5-fluorouracil for the treatment of a disorder.

Further provided by this aspect is a kit comprising a medicament comprising Gemcitabine or 5-fluorouracil and a package insert comprising instructions for administration of the medicament to an individual in combination with a composition comprising an ADC for the treatment of a disorder.

The disorder may be a proliferative disease, for example a cancer such as Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD25-ADC, such as ADCX25 described herein.

The ADC may be anti-CD25-ADC, such as ADCT-301.

The individual may be human. The individual may have cancer, or may have been determined to have cancer. The individual may have, or have been determined to have, a CD25+ cancer or CD25+ tumour-associated non-tumour cells, such as CD25+ infiltrating T-cells.

The individual may have, or have been determined to have, a PD-L1+ cancer.

The medicament or composition comprising the ADC may be administered before the medicament or composition comprising Gemcitabine or 5-fluorouracil, simultaneous with the medicament or composition comprising Gemcitabine or 5-fluorouracil, or after the medicament or composition comprising Gemcitabine or 5-fluorouracil. The treatment may comprise administering a further chemotherapeutic agent to the individual.

In a yet further aspect, the disclosure provides a composition comprising an ADC and Gemcitabine or 5-fluorouracil.

Also provided in this aspect of the disclosure is a method of treating a disorder in an individual, the method comprising administering to the individual an effective amount of the composition comprising an ADC and Gemcitabine or 5-fluorouracil.

Also provided in this aspect of the disclosure is a composition comprising an ADC and Gemcitabine or 5-fluorouracil for use in a method of treating a disorder in an individual.

Also provided in this aspect of the disclosure is the use of a composition comprising an ADC and Gemcitabine or 5-fluorouracil in the manufacture of a medicament for treating a disorder in an individual.

Also provided in this aspect of the disclosure is a kit comprising composition comprising an ADC and Gemcitabine or 5-fluorouracil and a set of instructions for administration of the medicament to an individual for the treatment of a disorder.

The disorder may be a proliferative disease, for example a cancer such as Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL), and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The ADC may be anti-CD25-ADC, such as ADCX25 described herein.

The ADC may be anti-CD25-ADC, such as ADCT-301.

The individual may be human. The individual may have cancer, or may have been determined to have cancer. The individual may have, or have been determined to have, a CD25+ cancer or CD25+ tumour-associated non-tumour cells, such as CD25+ infiltrating T-cells.

The individual may have, or have been determined to have, a PD-L1+ cancer.

The treatment may comprise administering a further chemotherapeutic agent to the individual.

### BRIEF DESCRIPTION OF THE FIGURES

Embodiments and experiments illustrating the principles of the disclosure will now be discussed with reference to the accompanying figures in which:

FIG. 1. Sequences

FIG. 2. In vivo efficacy study testing combination of sur301 with gemcitabine in the CT26 syngeneic model

FIG. 3. Schematic diagram of dose escalation. A perceived safe starting dose of 33% of the intended efficacious dose is proposed for both compounds, but this may need adaptation to lower or higher, as the individual risk profile for the combination may be. Compound 1 should be the compound for which an efficacious clinical dose has been firmly established (at 100%), and which is therefore aimed to be reached quickly in the trial patients by first escalating the dose of this compound.

### DETAILED DESCRIPTION

Antibody Drug Conjugates (ADCs)

The present disclosure relates to the improved efficacy of combinations of an ADC and Gemcitabine or 5-fluorouracil.

The ADC can deliver a drug to a target location. The target location is preferably a proliferative cell population. The antibody is an antibody for an antigen present on a proliferative cell population. In one aspect the antigen is absent or present at a reduced level in a non-proliferative cell population compared to the amount of antigen present in the proliferative cell population, for example a tumour cell population.

The ADC may comprise a linker which may be cleaved so as to release the drug at the target location. The drug may be a compound selected from RelA, RelB, RelC, RelD or RelE. Thus, the conjugate may be used to selectively provide a compound RelA, RelB, Rel C, RelD or RelE to the target location.

The linker may be cleaved by an enzyme present at the target location.

The disclosure particularly relates treatment with an anti-CD25 ADC disclosed in WO2014/057119, and as herein described.

Anti-CD25 ADCs

As used herein, the term “CD25-ADC” refers to an ADC in which the antibody component is an anti-CD25 antibody. The term “PBD-ADC” refers to an ADC in which the drug component is a pyrrolobenzodiazepine (PBD) warhead. The term “anti-CD25-ADC” refers to an ADC in which the antibody component is an anti-CD25 antibody, and the drug component is a PBD warhead.

The ADC may comprise a conjugate of formula L-(D^{L})ₚ, where D^{L} is of formula I or II:

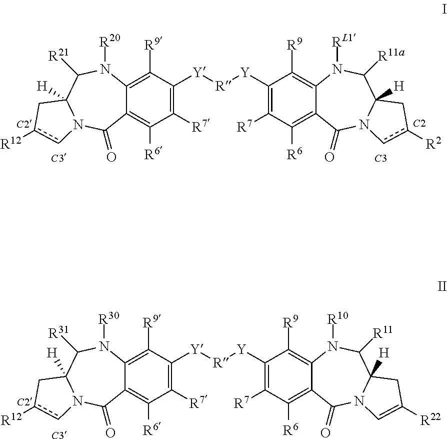

wherein:

L is an antibody (Ab) which is an antibody that binds to CD25;
when there is a double bond present between C2′ and C3′, R¹² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

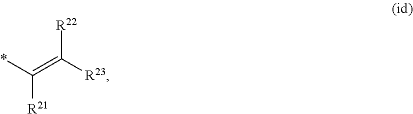

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

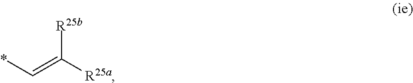

wherein one of R^{25a} and R^{25b} is H and the other is selected from: phenyl, which phenyl is optionally substituted by a group selected from halo, methyl, methoxy; pyridyl; and thiophenyl; and

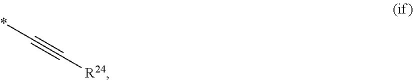

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2′ and C3′,

R¹² is

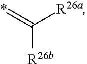

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;

R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NHRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{6′}, R^{7′}, R^{9′} are selected from the same groups as R⁶, R⁷ and R⁹ respectively;

[Formula I]

R^{L1′} is a linker for connection to the antibody (Ab);

R^{11a} is selected from OH, OR^{A}, where R^{A} is C₁₋₄ alkyl, and SO₂M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{C}, where R^{C} is a capping group;

R²¹ is selected from OH, OR^{A} and SO_{z}M;

when there is a double bond present between C₂ and C₃, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

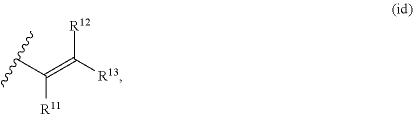

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

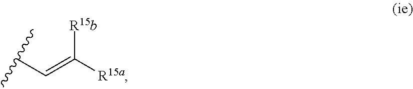

wherein one of R^{15a} and R^{15b} is H and the other is selected from: phenyl, which phenyl is optionally substituted by a group selected from halo, methyl, methoxy; pyridyl; and thiophenyl; and

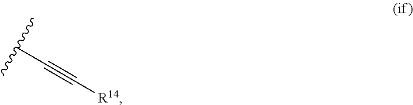

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,

R² is

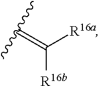

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

[Formula II]

R²² is of formula IIIa, formula IIIb or formula IIIc:

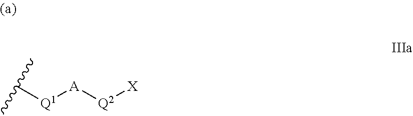

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

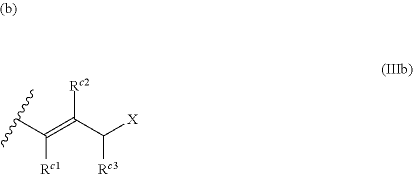

where;

R^{C1}, R^{C2} and R^{C3} are independently selected from H and unsubstituted C₁₋₂ alkyl;

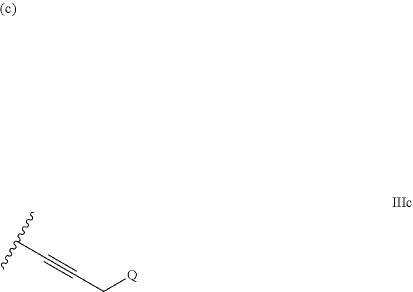

where Q is selected from O—R^{L2}'S—R^{L2′} and NR^{N}—R^{L2′}, and RN is selected from H, methyl and ethyl

X is selected from the group comprising: O—R^{L2′}, S—R^{L2′}, CO₂—R^{L2′}, CO—R^{L2′}, NH—C(═O)—R^{L2′},

NHNH—R^{L2′}, CONHNH—R^{L2′},

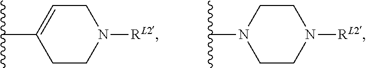

NR^{N}R^{L2′}, wherein R^{N} is selected from the group comprising H and C₁₋₄ alkyl;

R^{L2′} is a linker for connection to the antibody (Ab);

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M.

In some embodiments L-R^{L1′} or L-R^{L2′} is a group:

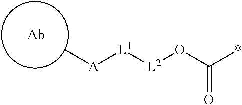

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

In some of these embodiments, L¹ is enzyme cleavable.

It has previously been shown that such ADCs are useful in the treatment of CD25 expressing cancers (see, for example, WO2014/057119, which is incorporated by reference herein in its entirety).

The term anti-CD25-ADC may include any embodiment described in WO 2014/057119. In particular, in preferred embodiments the ADC may have the chemical structure:

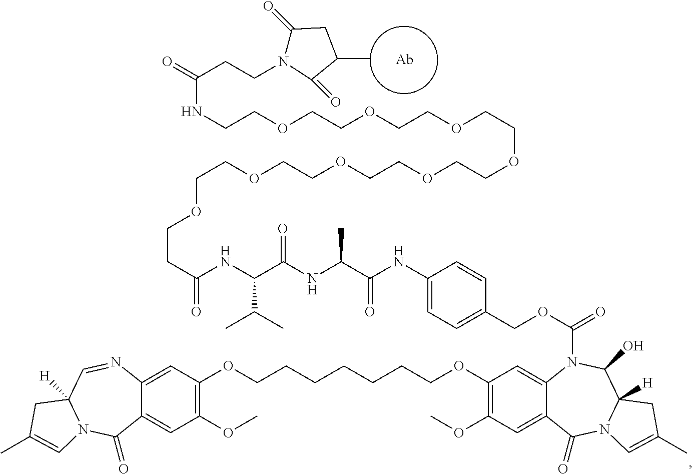

where the Ab is a CD25 antibody, and the DAR is between 1 and 8.

The antibody may comprise a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5.

In some aspects the antibody component of the anti-CD25-ADC is an antibody comprising: a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5. In some embodiments the antibody comprises a VH domain having the sequence according to SEQ ID NO. 1.

The antibody may further comprise: a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8. In some embodiments the antibody further comprises a VL domain having the sequence according to SEQ ID NO. 2.

In some embodiments the antibody comprises a VH domain and a VL domain, the VH and VL domains having the sequences of SEQ ID NO. 1 paired with SEQ ID NO. 2.

The VH and VL domain(s) may pair so as to form an antibody antigen binding site that binds CD25.

In preferred embodiments the antibody is an intact antibody comprising a VH domain and a VL domain, the VH and VL domains having sequences of SEQ ID NO. 1 and SEQ ID NO. 2.

In some embodiments the antibody is a fully human monoclonal IgG1 antibody, preferably IgG1,κ.

In some embodiments the antibody is the AB12 antibody described in WO 2004/045512 (Genmab A/S).

In an aspect the antibody is an antibody as described herein which has been modified (or further modified) as described below. In some embodiments the antibody is a humanised, deimmunised or resurfaced version of an antibody disclosed herein.

The preferred anti-CD25-ADC for use with the aspects of the present disclosure is ADCX25, as described herein below. A second preferred ant-CD25-ADC is ADCT-301.

ADC×25

ADC×25 is an antibody drug conjugate composed of a human antibody against human CD25 attached to a pyrrolobenzodiazepine (PBD) warhead via a cleavable linker. The mechanism of action of ADCX25 depends on CD25 binding. The CD25 specific antibody targets the antibody drug conjugate (ADC) to cells expressing CD25. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

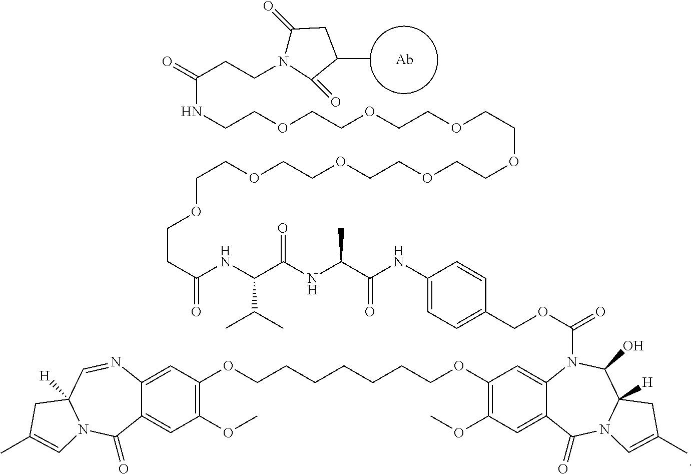

Ab represents Antibody AB12 (fully human monoclonal IgG1, K antibody with the VH and VL sequences SEQ ID NO. 1 and SEQ ID NO. 2, respectively, also known as HuMax-TAC). It is synthesised as described in WO 2014/057119 (Conj AB12-E) and typically has a DAR (Drug to Antibody Ratio) of 2.0+/−0.3.

CD25 Binding

The “first target protein” (FTP) as used herein is preferably CD25.

As used herein, “binds CD25” is used to mean the antibody binds CD25 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 104, 105 or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589.

Gemcitabine

Combination of agents with different action mechanisms is an established therapeutic principle for combating cancer. It can be a way of increasing anti-tumour activity when a synergic effect is shown and/or when reduced toxicity is observed. Antibody-drug conjugates, including those with a PBD warhead, may be particularly suited as combination partners because they are more targeted compared to conventional chemotherapy. As PBD dimers cross-link DNA in a covalent fashion, combining them with other agents that interfere with DNA synthesis via a different mechanism is likely to provide a benefit. An example combination is Gemcitabine.

Gemcitabine is a broad-spectrum antimetabolite and deoxycytidine analogue with antineoplastic activity. Upon administration, gemcitabine is converted into the active metabolites difluorodeoxycytidine diphosphate (dFdCDP) and difluorodeoxycytidine triphosphate (dFdCTP) by deoxycytidine kinase. dFdCTP competes with deoxycytidine triphosphate (dCTP) and is incorporated into DNA. This locks DNA polymerase thereby resulting in masked termination during DNA replication. On the other hand, dFdCDP inhibits ribonucleotide reductase, thereby decreasing the deoxynucleotide pool available for DNA synthesis. The reduction in the intracellular concentration of dCTP potentiates the incorporation of dFdCTP into DNA.

Gemcitabine has shown activity in a variety of solid tumors, and has been approved for the treatment of non-small cell lung cancer, pancreatic, bladder, and breast cancer. Recent data showed that gemcitabine is also active against ovarian cancer. Gemcitabine is reported to have a good toxicity profile, with myelosuppression being the most common side effect, while non-hematological events are relatively uncommon (Toschi et al. 2005, Future Oncology, Vol. 1(1), pp. 7-17).
i. CAS Number→95058-81-4
   (see cas.org/content/chemical-substances/faqs)
ii. IUPHAR/BPS reference→4793
   (see guidetopharmacology.org/)
iii. Unique Ingredient Identifier (UNII)→B76N6SBZ8R
(see fda.gov/Forlndustry/DataStandards/SubstanceRegistrationSystem-UniquelngredientIdentifierUNll/default.htm)

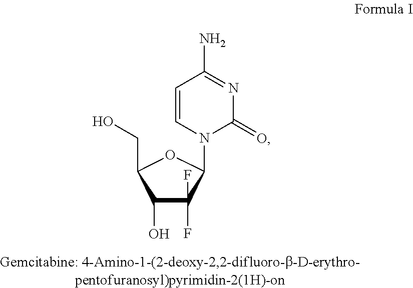

5-fluorouracil

Combination of agents with different action mechanisms is an established therapeutic principle for combating cancer. It can be a way of increasing anti-tumour activity when a synergic effect is shown and/or when reduced toxicity is observed. Antibody-drug conjugates, including those with a PBD warhead, may be particularly suited as combination partners because they are more targeted compared to conventional chemotherapy. As PBD dimers cross-link DNA in a covalent fashion, combining them with other agents that interfere with DNA synthesis via a different mechanism is likely to provide a benefit. Another example combination is 5-fluorouracil

5-fluorouracil is an antimetabolite fluoropyrimidine analog of the nucleoside pyrimidine with antineoplastic activity. 5-fluorouracil and its metabolites possess a number of different mechanisms of action. In vivo, 5-fluoruracil is converted to the active metabolite 5-fluoroxyuridine monophosphate (F-UMP); replacing uracil, F-UMP incorporates into RNA and inhibits RNA processing, thereby inhibiting cell growth. Another active metabolite, 5-5-fluoro-2′-deoxyuridine-5′-O-monophosphate (F-dUMP), inhibits thymidylate synthase, resulting in the depletion of thymidine triphosphate (TTP), one of the four nucleotide triphosphates used in the in vivo synthesis of DNA. Other

5-fluorouracil metabolites incorporate into both RNA and DNA; incorporation into RNA results in major effects on both RNA processing and functions. 5-fluorouracil has shown activity in a variety of solid tumors, and has been approved for the treatment of anal, breast, colorectal, oesophageal, stomach, pancreatic and skin cancers (especially head and neck cancers). It has also been given topically for actinic keratoses, skin cancers and Bowen's disease and as eye drops for treatment of ocular surface squamous neoplasia. Other uses include ocular injections into a previously created trabeculectomy bleb to inhibit healing and cause scarring of tissue, thus allowing adequate aqueous humor flow to reduce intraocular pressure.
i. CAS Number→51-21-8
   (see cas.org/content/chemical-substances/faqs)
ii. IUPHAR/BPS reference→4789
   (see guidetopharmacology.org/)
iii. Unique Ingredient Identifier (UNII)→U3P01618RT
(see fda.gov/Forlndustry/DataStandards/SubstanceRegistrationSystem-UniquelngredientIdentifierUNII/default.htm)

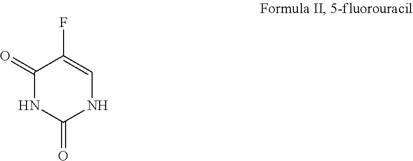

Advantageous Properties of the Described Combination

Both the ADC and Gemcitabine or 5-fluorouracil when used as a single agent in isolation have demonstrated clinical utility—for example, in the treatment of cancer. However, as described herein, combination of the ADC and Gemcitabine or 5-fluorouracil is expected to provide one or more of the following advantages over treatment with either ADC or Gemcitabine or 5-fluorouracil alone:
1) effective treatment of a broader range of cancers;
2) effective treatment of resistant or refractory forms of disorders such as cancer, and individuals with disorders such as cancer who have relapsed after a period of remission;
3) increased response rate to treatment; and/or
4) Increased durability of treatment.

Effective treatment of a broader range of cancers as used herein means that following treatment with the combination a complete response is observed with a greater range of recognised cancer types. That is, a complete response is seen from cancer types not previously reported to completely respond to either ADC or Gemcitabine or 5-fluorouracil alone.

Effective treatment of a resistant, refractory, or relapsed forms as used herein means that following treatment with the combination a complete response is observed in individuals that are either partially or completely resistant or refractory to treatment with either ADC or Gemcitabine or 5-fluorouracil alone (for example, individuals who show no response or only partial response following treatment with either agent alone, or those with relapsed disorder). In some embodiments, a complete response following treatment with the ADC/Gemcitabine or 5-fluorouracil combination is observed at least 10% of individuals that are either partially or completely resistant or refractory to treatment with either ADC or Gemcitabine or 5-fluorouracil alone. In some embodiments, a complete response following treatment with the ADC/Gemcitabine or 5-fluorouracil combination is observed at least 20%, at least 30%, at least 40%, at least 50%, at least 60%, at least 70%, at least 80%, at least 90%, at least 95%, at least 98%, or at least 99% of individuals that are either partially or completely resistant or refractory to treatment with either ADC or Gemcitabine or 5-fluorouracil alone.

Increased response rate to treatment as used herein means that following treatment with the combination a complete response is observed in a greater proportion of individuals than is observed following treatment with either ADC or Gemcitabine or 5-fluorouracil alone. In some embodiments, a complete response following treatment with the ADC/Gemcitabine or 5-fluorouracil combination is observed at least 10% of treated individuals.

In some embodiments, a complete response following treatment with the ADC/Gemcitabine or 5-fluorouracil combination is observed at least 20%, at least 30%, at least 40%, at least 50%, at least 60%, at least 70%, at least 80%, at least 90%, at least 95%, at least 98%, or at least 99% of treated individuals.

Increased durability of treatment as used herein means that average duration of complete response in individuals treated with the combination is longer than in individuals who achieve complete response following treatment with either ADC or Gemcitabine or 5-fluorouracil alone. In some embodiments, the average duration of a complete response following treatment with the ADC/Gemcitabine or 5-fluorouracil combination is at least 6 months. In some embodiments, the average duration of a complete response following treatment with the ADC/Gemcitabine or 5-fluorouracil combination is at least 12 months, at least 18 months, at least 24 months, at least 3 years, at least 4 years, at least 5 years, at least 6 years, at least 7 years, at least 8 years, at least 9 years, at least 10 years, at least 15 years, or at least 20 years.

‘Complete response’ is used herein to mean the absence of any clinical evidence of disease in an individual. Evidence may be assessed using the appropriate methodology in the art, for example CT or PET scanning, or biopsy where appropriate. The number of doses required to achieve complete response may be one, two, three, four, five, ten or more. In some embodiments the individuals achieve complete response no more than a year after administration of the first dose, such as no more than 6 months, no more than 3 months, no more than a month, no more than a fortnight, or no more than a week after administration of the first dose.

Treated Disorders

The combined therapies described herein include those with utility for anticancer activity. In particular, in certain aspects the therapies include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides combined therapies comprising administering an ADC which binds a first target protein for use in therapy, wherein the method comprises selecting a subject based on expression of the target protein.

In one aspect, the present disclosure provides a combined therapy with a label that specifies that the therapy is suitable for use with a subject determined to be suitable for such use. The label may specify that the therapy is suitable for use in a subject has expression of the first target protein, such as overexpression of the first target protein. The label may specify that the subject has a particular type of cancer.

The first target protein is preferably CD25. The cancer may be lymphoma, such as AML. The label may specify that the subject has a CD25+ lymphoma.

In a further aspect there is also provided a combined therapy as described herein for use in the treatment of a proliferative disease. Another aspect of the present disclosure provides the use of a conjugate compound in the manufacture of a medicament for treating a proliferative disease.

One of ordinary skill in the art is readily able to determine whether or not a candidate combined therapy treats a proliferative condition for any particular cell type. For example, assays which may conveniently be used to assess the activity offered by a particular compound are described below.

The combined therapies described herein may be used to treat a proliferative disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Proliferative disorders of particular interest include, but are not limited to, Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12].

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

It is contemplated that the combined therapies of the present disclosure may be used to treat various diseases or disorders, e.g. characterized by the overexpression of a tumor antigen. Exemplary conditions or hyperproliferative disorders include benign or malignant tumors; leukemia, haematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which the combined therapies may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, graft-versus-host disease (GVHD), and autoimmune endocrine disorders (such as, for example, diabetic-related autoimmune diseases such as insulin-dependent diabetes mellitus (IDDM), Addison's disease, and autoimmune thyroid disease (e.g. Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

In some aspects, the subject has a proliferative disorder selected from (classical) Hodgkin lymphomas, with mixed cellularity type (Hodgkin-/Reed-Sternbert-Cells: CD25 +/−), or non-Hodgkin lymphoma, including B-cell chronic lymphatic leukemia, diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12], small cell lymphocytic lymphoma, adult T-cell leukemia/lymphoma, or anaplastic large cell lymphoma.

In some aspects, the subject has a proliferative disease characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells.

The target neoplasm or neoplastic cells may be all or part of a solid tumour.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25−ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

Classical Hodgkins lymphoma includes the subtypes nodular sclerosing, lymphocyte predominant, lymphocyte depleted and mixed cellularity. The Hodgkins lymphoma subtype may not be defined. In certain aspects, the patients tested according to the methods here have Hodgkins lymphoma of the nodular sclerosing and mixed cellularity subtypes.

In certain aspects, the subject has diffuse large B cell lymphoma or peripheral T cell lymphoma, including the anaplastic large cell lymphoma and angioimmunoblastic T cell lymphoma subtypes.

Patient Selection

In certain aspects, the individuals are selected as suitable for treatment with the combined treatments before the treatments are administered.

As used herein, individuals who are considered suitable for treatment are those individuals who are expected to benefit from, or respond to, the treatment. Individuals may have, or be suspected of having, or be at risk of having cancer. Individuals may have received a diagnosis of cancer. In particular, individuals may have, or be suspected of having, or be at risk of having, lymphoma. In some cases, individuals may have, or be suspected of having, or be at risk of having, a solid cancer that has tumour associated non-tumor cells that express a first target protein, such as infiltrating T-cells that express a first target protein.

In some aspects, individuals are selected on the basis of the amount or pattern of expression of a first target protein. In some aspects, the selection is based on expression of a first target protein at the cell surface. So, in some cases, individuals are selected on the basis they have, or are suspected of having, are at risk of having cancer, or have received a diagnosis of a proliferative disease characterised by the presence of a neoplasm comprising cells having a low level of surface expression of a first target protein (such as CD25). The neoplasm may be composed of cells having a low level of surface expression of a first target protein (such as CD25). In some cases, low levels of surface expression means that mean number of anti-FTP antibodies bound per neoplastic cell is less than 20000, such as less than 10000, less than 5000, less than 2000, less than 1000, less than 500, less than 400, less than 300, less than 200, or less than 100. In some cases, the mean number of bound antibodies per cell is measured using the assay described in Example 9.

In some aspects, individuals are selected on the basis they have a neoplasm comprising both CD25+ve and CD25−ve cells. The neoplasm may be composed of CD25−ve neoplastic cells, optionally wherein the CD25−ve neoplastic cells are associated with CD25+ve non-neoplastic cells such as CD25+ve T-cells. The neoplasm or neoplastic cells may be all or part of a solid tumour. The solid tumour may be partially or wholly CD25−ve, and may be infiltrated with CD25+ve cells, such as CD25+ve T-cells.

In certain aspects, the target is a second target protein. In some aspects, the selection is based on expression of a second target protein at the cell surface.

In some aspects, the selection is based on levels of both a first target protein and a second target protein at the cell surface.

In some cases, expression of the target in a particular tissue of interest is determined. For example, in a sample of lymphoid tissue or tumor tissue. In some cases, systemic expression of the target is determined. For example, in a sample of circulating fluid such as blood, plasma, serum or lymph.

In some aspects, the individual is selected as suitable for treatment due to the presence of target expression in a sample. In those cases, individuals without target expression may be considered not suitable for treatment.

In other aspects, the level of target expression is used to select a individual as suitable for treatment. Where the level of expression of the target is above a threshold level, the individual is determined to be suitable for treatment.

In some aspects, the presence of a first target protein and/or a second target protein in cells in the sample indicates that the individual is suitable for treatment with a combination comprising an ADC and Gemcitabine or 5-fluorouracil. In other aspects, the amount of first target protein and/or a second target protein expression must be above a threshold level to indicate that the individual is suitable for treatment. In some aspects, the observation that first target protein and/or a second target protein localisation is altered in the sample as compared to a control indicates that the individual is suitable for treatment.

In some aspects, an individual is indicated as suitable for treatment if cells obtained from lymph node or extra nodal sites react with antibodies against first target protein and/or a second target protein as determined by IHC.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a first target protein. In some aspects disclosed herein, a patient is determined to be suitable for treatment if at least at least 10% of the cells in the sample express a first target protein.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a second target protein. In some aspects disclosed herein, a patient is determined to be suitable for treatment if at least at least 10% of the cells in the sample express a second target protein.

The first target protein is preferably CD25.

Samples

The sample may comprise or may be derived from: a quantity of blood; a quantity of serum derived from the individual's blood which may comprise the fluid portion of the blood obtained after removal of the fibrin clot and blood cells; a quantity of pancreatic juice; a tissue sample or biopsy; or cells isolated from said individual.

A sample may be taken from any tissue or bodily fluid. In certain aspects, the sample may include or may be derived from a tissue sample, biopsy, resection or isolated cells from said individual.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a first target polypeptide or nucleic acid in the blood, in white blood cells, peripheral blood mononuclear cells, granulocytes and/or red blood cells.

The sample may be fresh or archival. For example, archival tissue may be from the first diagnosis of an individual, or a biopsy at a relapse. In certain aspects, the sample is a fresh biopsy.

The first target polypeptide is preferably CD25.

Individual Status

The individual may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the individual may be any of its forms of development, for example, a foetus. In one preferred embodiment, the individual is a human. The terms “subject”, “patient” and “individual” are used interchangeably herein.

In some aspects disclosed herein, an individual has, or is suspected as having, or has been identified as being at risk of, cancer. In some aspects disclosed herein, the individual has already received a diagnosis of cancer. The individual may have received a diagnosis of (classical) Hodgkins lymphoma (including nodular sclerosing, lymphocyte predominant, lymphocyte, or mixed cellularity type, or where the type is unspecified), diffuse large B cell lymphoma (DLBCL) or peripheral T cell lymphoma (PTCL) (including the subtypes ALCL: anaplastic large cell lymphoma or AITL: angioimmunoblastic T cell lymphoma). In some cases, the individual has received a diagnosis of nodular sclerosing or mixed cellularlity classical Hodgkins lymphoma, diffuse large B cell lymphoma, or angioimmunoblastic T cell lymphoma.

In some cases the individual has, or is suspected of having, a proliferative disease characterised by the presence of a neoplasm comprising both CD25+ve and CD25−ve cells. The neoplasm may be composed of CD25−ve neoplastic cells, optionally wherein the CD25−ve neoplastic cells are associated with CD25+ve non-neoplastic cells such as CD25+ve T-cells. The neoplasm or neoplastic cells may be all or part of a solid tumour.

The solid tumor may be a neoplasm, including a non-haematological cancer, comprising or composed of CD25+ve neoplastic cells. The solid tumor may be a neoplasm, including a non-haematological cancer, infiltrated with CD25+ve cells, such as CD25+ve T-cells; such solid tumours may lack expression of CD25 (that is, comprise or be composed of CD25−ve neoplastic cells).

In some cases the individual has, or is suspected of having, a solid tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). The some or all of the neoplastic cells in the tumour may be CD25−ve. The solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

In some cases, the individual has received a diagnosis of (classical) Hodgkins lymphoma (mixed cellularity type), or non-Hodgkins lymphoma (including B-cell chronic lymphatic leukemia, diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12], small cell lymphocytic lymphoma, adult T-cell leukemia/lymphoma, and anaplastic large cell lymphoma.

In some cases, the individual has received a diagnosis of cutaneous T-cell lymphoma, mycosis fungoides, Sezary syndrome, systemic mastocytosis, B-cell lymphoma, non-hematopoietic tumors, peripheral T cell lymphoma and histiocytic proliferation.

In some cases, the individuals has, or is suspected of having, or has received a diagnosis of a proliferative disease characterised by the presence of a neoplasm comprising cells having a low level of surface expression of a first target protein (such as CD25). The neoplasm may be composed of cells having a low level of surface expression of a first target protein (such as CD25). In some cases, low levels of surface expression means that mean number of anti-FTP antibodies bound per neoplastic cell is less than 20000, such as less than 10000, less than 5000, less than 2000, less than 1000, less than 500, less than 400, less than 300, less than 200, or less than 100.

In some cases, the individual has received a diagnosis of a proliferative disease characterised by the presence of a neoplasm comprising both CD25+ve and CD25−ve cells. The neoplasm may be composed of CD25−ve neoplastic cells, optionally wherein the CD25−ve neoplastic cells are associated with CD25+ve non-neoplastic cells such as CD25+ve T-cells. The neoplasm or neoplastic cells may be all or part of a solid tumour. The solid tumor may be a neoplasm, including a non-haematological cancer, comprising or composed of CD25+ve neoplastic cells. The solid tumor may be a neoplasm, including a non-haematological cancer, infiltrated with CD25+ve cells, such as CD25+ve T-cells; such solid tumours may lack expression of CD25 (that is, comprise or be composed of CD25−ve neoplastic cells).

In some cases, the individual has received a diagnosis of a solid tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27(1):109-118). The some or all of the neoplastic cells in the tumour may be CD25−ve. The solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

In some cases, the individual has received a diagnosis of a solid cancer containing CD25+ expressing infiltrating T-cells.

The Individual may be undergoing, or have undergone, a therapeutic treatment for that cancer. The subject may, or may not, have previously received ADCX25 or ADCT-301. In some cases the cancer is lymphoma, including Hodgkins or non-Hodgkins lymphoma.

Controls

In some aspects, target expression in the individual is compared to target expression in a control. Controls are useful to support the validity of staining, and to identify experimental artefacts.

In some cases, the control may be a reference sample or reference dataset. The reference may be a sample that has been previously obtained from a individual with a known degree of suitability. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from individuals who are known to benefit from the treatment. The tissue may be of the same type as the sample being tested. For example, a sample of tumor tissue from a individual may be compared to a control sample of tumor tissue from a individual who is known to be suitable for the treatment, such as a individual who has previously responded to the treatment.

In some cases the control may be a sample obtained from the same individual as the test sample, but from a tissue known to be healthy. Thus, a sample of cancerous tissue from a individual may be compared to a non-cancerous tissue sample.

In some cases, the control is a cell culture sample.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern.

For example, where the amount of a first target protein and/or a second target protein expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

The first target protein is preferably CD25.

Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC and Gemcitabine or 5-fluorouracil. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The ADC may comprise an anti-CD25 antibody. The anti-CD25 antibody may be HuMax-TAC™. The ADC may comprise a drug which is a PBD dimer. The ADC may be a anti-CD25-ADC, and in particular, ADCX25 or ADCT-301. The ADC may be an ADC disclosed in WO2014/057119.

The treatment may involve administration of the ADC/Gemcitabine or 5-fluorouracil combination alone or in further combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1l, calicheamicin omegal1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C₄, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Dosage

It will be appreciated by one of skill in the art that appropriate dosages of the ADC and/or Gemcitabine or 5-fluorouracil, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of a first target protein observed in a sample obtained from the subject. Thus, the level or localisation of expression of the first target protein in the sample may be indicative that a higher or lower dose of ADC is required. For example, a high expression level of the first target protein may indicate that a higher dose of ADC would be suitable. In some cases, a high expression level of the first target protein may indicate the need for administration of another agent in addition to the ADC. For example, administration of the ADC in conjunction with a chemotherapeutic agent. A high expression level of the first target protein may indicate a more aggressive therapy.

In certain aspects, the dosage level is determined by the expression of a first target protein on neoplastic cells in a sample obtained from the subject. For example, when the target neoplasm is composed of, or comprises, neoplastic cells expressing the first target protein.

In certain aspects, the dosage level is determined by the expression of a first target protein on cells associated with the target neoplasm. For example, the target neoplasm may be a solid tumour composed of, or comprising, neoplastic cells that express the first target protein. For example, the target neoplasm may be a solid tumour composed of, or comprising, neoplastic cells that do not express the first target protein. The cells expressing the first target protein may be non-neoplastic cells infiltrating the solid tumour, such as infiltrating T-cells.

In certain aspects, the dosage of Gemcitabine or 5-fluorouracil is determined by the expression of a second target protein observed in a sample obtained from the subject. Thus, the level or localisation of expression of the second target protein in the sample may be indicative that a higher or lower dose of Gemcitabine or 5-fluorouracil is required. For example, a high expression level of the second target protein may indicate that a higher dose of Gemcitabine or 5-fluorouracil would be suitable. In some cases, a high expression level of the second target protein may indicate the need for administration of another agent in addition to Gemcitabine or 5-fluorouracil. For example, administration of Gemcitabine or 5-fluorouracil in conjunction with a chemotherapeutic agent. A high expression level of the second target protein may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

For the ADC, where it is a PBD bearing ADC, the dosage amounts described above may apply to the conjugate (including the PBD moiety and the linker to the antibody) or to the effective amount of PBD compound provided, for example the amount of compound that is releasable after cleavage of the linker.

The first target protein is preferably CD25. The ADC may comprise an anti-CD25 antibody. The anti-CD25 antibody may be HuMax-TAC™. The ADC may comprise a drug which is a PBD dimer. The ADC may be an anti-CD25-ADC, and in particular, is preferably ADCX25 or ADCT-301. The ADC may be an ADC disclosed in WO2014/057119.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

Anti-CD25 antibodies are known in the art and are useful in the methods disclosed herein. These include antibodies 4C9 (obtainable from Ventana Medical Systems, Inc.). Other suitable antibodies include antibody AB12 described in WO 2004/045512 (Genmab A/S), IL2R.1 (obtainable from Life Technologies, catalogue number MA5-12680) and RFT5 (described in U.S. Pat. No. 6,383,487). Other suitable antibodies include B489 (143-13) (obtainable from Life Technologies, catalogue number MA1-91221), SP176 (obtainable from Novus, catalogue number NBP2-21755), 1B5D12 (obtainable from Novus, catalogue number NBP2-37349), 2R12 (obtainable from Novus, catalogue number NBP2-21755), or BC96 (obtainable from BioLegend, catalogue number V T-072) and M-A251 (obtainable from BioLegend, catalogue number IV A053). Other suitable anti-CD25 antibodies are daclizumab (Zenapax™) and basiliximab (Simulect™), both of which have been approved for clinical use.

Anti-PD-L1 antibodies are known in the art and are useful in the methods disclosed herein. These antibodies include Atezolizumab (MPDL3280; CAS number 1380723-44-3), Avelumab (MSB0010718C; CAS number 1537032-82-8), and Durvalumab (CAS number 1428935-60-7).

### BRIEF DESCRIPTION OF THE FIGURES

Embodiments and experiments illustrating the principles of the disclosure will now be discussed with reference to the accompanying figures in which:

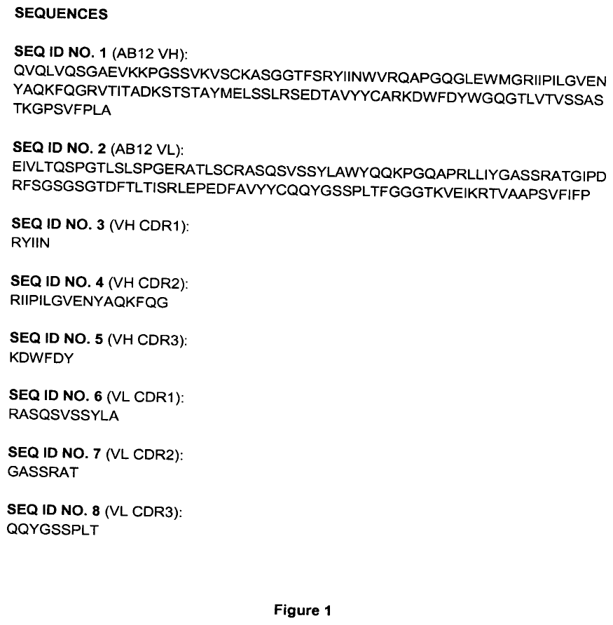

FIG. 1. Sequences

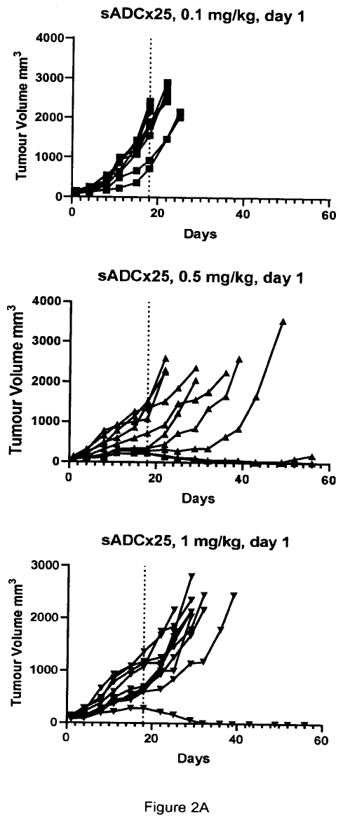

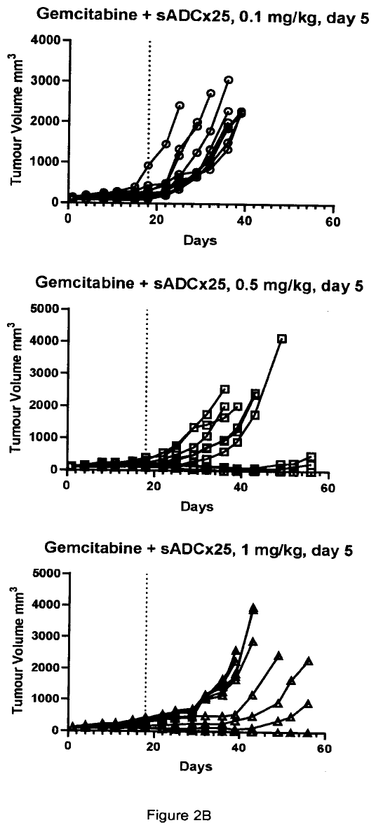

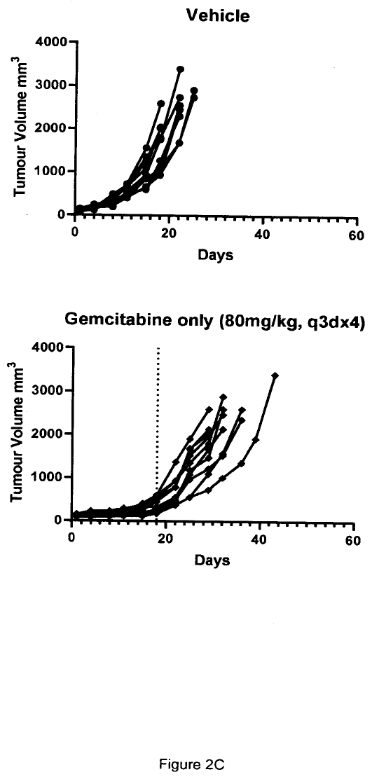

FIG. 2. In vivo efficacy study testing combination of sur301 with gemcitabine in the CT26 syngeneic model

The disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value.

Similarly, when values are expressed as approximations, by the use of the antecedent “about,” it will be understood that the particular value forms another embodiment.

### Some Embodiments

The following paragraphs describe some specific embodiments of the present disclosure:

1. A method for treating cancer in an individual, the method comprising administering to the individual an effective amount of ADCX25 or ADCT-301 and Gemcitabine.

2. A first composition comprising ADCX25 or ADCT-301 for use in a method of treating cancer in an individual, wherein the treatment comprises administration of the first composition in combination with a second composition comprising Gemcitabine.

3. A first composition comprising Gemcitabine for use in a method of treating a disorder in an individual, wherein the treatment comprises administration of the first composition in combination with a second composition comprising ADCX25 or ADCT-301.

4. Use of ADCX25 or ADCT-301 in the manufacture of a medicament for treating cancer in an individual, wherein the medicament comprises ADCX25 or ADCT-301, and wherein the treatment comprises administration of the medicament in combination with a composition comprising Gemcitabine.

5. Use of a Gemcitabine in the manufacture of a medicament for treating cancer in an individual, wherein the medicament comprises Gemcitabine, and wherein the treatment comprises administration of the medicament in combination with a composition comprising ADCX25 or ADCT-301.

6. A kit comprising:
a first medicament comprising ADCX25 or ADCT-301;
a second medicament comprising Gemcitabine; and, optionally,
a package insert comprising instructions for administration of the first medicament to an individual in combination with the second medicament for the treatment of cancer.

7. A kit comprising a medicament comprising ADCX25 or ADCT-301 and a package insert comprising instructions for administration of the medicament to an individual in combination with a composition comprising Gemcitabine for the treatment of cancer.

8. A kit comprising a medicament comprising Gemcitabine and a package insert comprising instructions for administration of the medicament to an individual in combination with a composition comprising ADCX25 or ADCT-301 for the treatment of cancer.

9. A pharmaceutical composition comprising ADCX25 or ADCT-301 and Gemcitabine.

10. A method of treating cancer in an individual, the method comprising administering to the individual an effective amount of the composition of paragraph 9.

11. The composition of paragraph 9 for use in a method of treating cancer in an individual.

12. The use of the composition of paragraph 9 in the manufacture of a medicament for treating cancer in an individual.

13. A kit comprising the composition of paragraph 9 and a set of instructions for administration of the medicament to an individual for the treatment of cancer.

14. The composition, method, use, or kit according to any previous paragraph, wherein the treatment comprises administering ADCX25 or ADCT-301 before Gemcitabine, simultaneous with Gemcitabine, or after Gemcitabine.

15. The composition, method, use, or kit according to any previous paragraph, wherein the treatment further comprises administering a chemotherapeutic agent.

16. The composition, method, use, or kit according to any previous paragraph, wherein the individual is human.

17. The composition, method, use, or kit according to any previous paragraph, wherein the individual has, or has been determined to have, cancer.

18. The composition, method, use, or kit according any previous paragraph, wherein the individual has, or has been has been determined to have, a cancer characterised by the presence of a neoplasm comprising both CD25+ve and CD25−ve cells.

19. The composition, method, use, or kit according any previous paragraph, wherein the individual has, or has been has been determined to have, a cancer characterised by the presence of a neoplasm comprising, or composed of, CD25−ve neoplastic cells.

20. The composition, method, use, or kit according to any previous paragraph, wherein the cancer or neoplasm is all or part of a solid tumour.

21. The composition, method, use, or kit according to any previous paragraph, wherein the individual has, or has been has been determined to have, a cancer which expresses CD25 or CD25+ tumour-associated non-tumour cells, such as CD25+ infiltrating T-cells.

22. The composition, method, use, or kit according to any previous paragraph, wherein the individual has, or has been has been determined to have, a cancer which expresses a low level of surface expression of CD25.

23. The composition, method, use, or kit according to any preceding paragraph, wherein the individual has, or has been has been determined to have, a cancer which expresses a second target protein.

24. The composition, method, use, or kit according to any one of the preceding paragraphs, wherein the treatment:
a) effectively treats a broader range of disorders,
b) effectively treats resistant, refractory, or relapsed disorders,
c) has an increased response rate, and/or
d) has increased durability;
as compared to treatment with either ADCX25 or ADCT-301 or Gemcitabine alone.

25. The composition, method, use, or kit according to any one of the preceding paragraphs, wherein the cancer is selected from the group comprising:
Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL);
   leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL);
pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

1a. A method for treating cancer in an individual, the method comprising administering to the individual an effective amount of ADCX25 or ADCT-301 and 5-fluorouracil.

2a. A first composition comprising ADCX25 or ADCT-301 for use in a method of treating cancer in an individual, wherein the treatment comprises administration of the first composition in combination with a second composition comprising 5-fluorouracil.

3a. A first composition comprising 5-fluorouracil for use in a method of treating a disorder in an individual, wherein the treatment comprises administration of the first composition in combination with a second composition comprising ADCX25 or ADCT-301.

4a. Use of ADCX25 or ADCT-301 in the manufacture of a medicament for treating cancer in an individual, wherein the medicament comprises ADCX25 or ADCT-301, and wherein the treatment comprises administration of the medicament in combination with a composition comprising 5-fluorouracil.

5a. Use of 5-fluorouracil in the manufacture of a medicament for treating cancer in an individual, wherein the medicament comprises 5-fluorouracil, and wherein the treatment comprises administration of the medicament in combination with a composition comprising ADCX25 or ADCT-301.

6a. A kit comprising:
a first medicament comprising ADCX25 or ADCT-301;
a second medicament comprising 5-fluorouracil; and, optionally,
a package insert comprising instructions for administration of the first medicament to an individual in combination with the second medicament for the treatment of cancer.

7a. A kit comprising a medicament comprising ADCX25 or ADCT-301 and a package insert comprising instructions for administration of the medicament to an individual in combination with a composition comprising 5-fluorouracil for the treatment of cancer.

8a. A kit comprising a medicament comprising 5-fluorouracil and a package insert comprising instructions for administration of the medicament to an individual in combination with a composition comprising ADCX25 or ADCT-301 for the treatment of cancer.

9a. A pharmaceutical composition comprising ADCX25 or ADCT-301 and 5-fluorouracil.

10a. A method of treating cancer in an individual, the method comprising administering to the individual an effective amount of the composition of paragraph 9a.

11a. The composition of paragraph 9 for use in a method of treating cancer in an individual.

12a. The use of the composition of paragraph 9a in the manufacture of a medicament for treating cancer in an individual.

13a. A kit comprising the composition of paragraph 9a and a set of instructions for administration of the medicament to an individual for the treatment of cancer.

14a. The composition, method, use, or kit according to any previous paragraph, wherein the treatment comprises administering ADCX25 or ADCT-301 before 5-fluorouracil, simultaneous with 5-fluorouracil, or after 5-fluorouracil.

15a. The composition, method, use, or kit according to any previous paragraph, wherein the treatment further comprises administering a chemotherapeutic agent.

16a. The composition, method, use, or kit according to any previous paragraph, wherein the individual is human.

17a. The composition, method, use, or kit according to any previous paragraph, wherein the individual has, or has been determined to have, cancer.

18a. The composition, method, use, or kit according any previous paragraph, wherein the individual has, or has been has been determined to have, a cancer characterised by the presence of a neoplasm comprising both CD25+ve and CD25−ve cells.

19a. The composition, method, use, or kit according any previous paragraph, wherein the individual has, or has been has been determined to have, a cancer characterised by the presence of a neoplasm comprising, or composed of, CD25−ve neoplastic cells.

20a. The composition, method, use, or kit according to any previous paragraph, wherein the cancer or neoplasm is all or part of a solid tumour.

21a. The composition, method, use, or kit according to any previous paragraph, wherein the individual has, or has been has been determined to have, a cancer which expresses CD25 or CD25+ tumour-associated non-tumour cells, such as CD25+ infiltrating T-cells.

22a. The composition, method, use, or kit according to any previous paragraph, wherein the individual has, or has been has been determined to have, a cancer which expresses a low level of surface expression of CD25.

23a. The composition, method, use, or kit according to any preceding paragraph, wherein the individual has, or has been has been determined to have, a cancer which expresses a second target protein.

24a. The composition, method, use, or kit according to any one of the preceding paragraphs, wherein the treatment:
a) effectively treats a broader range of disorders,
b) effectively treats resistant, refractory, or relapsed disorders,
c) has an increased response rate, and/or
d) has increased durability;
as compared to treatment with either ADCX25 or ADCT-301 or 5-fluorouracil alone.

25a. The composition, method, use, or kit according to any one of the preceding paragraphs, wherein the cancer is selected from the group comprising:
Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL);
   leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL);
pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

### STATEMENTS OF INVENTION

1. A method for treating a disorder in an individual, the method comprising administering to the individual an effective amount of an ADC and Gemcitabine.

2. A first composition comprising an ADC for use in a method of treating a disorder in an individual, wherein the treatment comprises administration of the first composition in combination with a second composition comprising Gemcitabine.

3. A first composition comprising Gemcitabine for use in a method of treating a disorder in an individual, wherein the treatment comprises administration of the first composition in combination with a second composition comprising an ADC.

4. Use of an ADC in the manufacture of a medicament for treating a disorder in an individual, wherein the medicament comprises an ADC, and wherein the treatment comprises administration of the medicament in combination with a composition comprising Gemcitabine.

5. Use of Gemcitabine in the manufacture of a medicament for treating a disorder in an individual, wherein the medicament comprises Gemcitabine, and wherein the treatment comprises administration of the medicament in combination with a composition comprising an ADC.

6. A kit comprising:
a first medicament comprising an ADC;
a second medicament comprising Gemcitabine; and, optionally,
a package insert comprising instructions for administration of the first medicament to an individual in combination with the second medicament for the treatment of a disorder.

7. A kit comprising a medicament comprising an ADC and a package insert comprising instructions for administration of the medicament to an individual in combination with a composition comprising Gemcitabine for the treatment of a disorder.

8. A kit comprising a medicament comprising Gemcitabine and a package insert comprising instructions for administration of the medicament to an individual in combination with a composition comprising an ADC for the treatment of a disorder.

9. A pharmaceutical composition comprising an ADC and Gemcitabine.

10. A method of treating a disorder in an individual, the method comprising administering to the individual an effective amount of the composition of paragraph 9.

11. The composition of paragraph 9 for use in a method of treating a disorder in an individual.

12. The use of the composition of paragraph 9 in the manufacture of a medicament for treating a disorder in an individual.

13. A kit comprising the composition of paragraph 9 and a set of instructions for administration of the medicament to an individual for the treatment of a disorder.

14. The composition, method, use, or kit according to any previous paragraph, wherein the treatment comprises administering the ADC before Gemcitabine, simultaneous with Gemcitabine, or after Gemcitabine.

15. The composition, method, use, or kit according to any previous paragraph, wherein the treatment further comprises administering a chemotherapeutic agent.

16. The composition, method, use, or kit according to any previous paragraph, wherein the individual is human.

17. The composition, method, use, or kit according to any preceding paragraph, wherein the individual has a disorder or has been determined to have a disorder.

18. The composition, method, use, or kit according to paragraph 17, wherein the individual has, or has been has been determined to have, a cancer which expresses CD25 or CD25+ tumour-associated non-tumour cells, such as CD25+ infiltrating T-cells.

19. The composition, method, use, or kit according to any one of the preceding paragraphs, wherein the treatment:
a) effectively treats a broader range of disorders,
b) effectively treats resistant, refractory, or relapsed disorders,
c) has an increased response rate, and/or
d) has increased durability;

as compared to treatment with either the ADC or Gemcitabine alone.

20. A composition, method, use, or kit according to any previous paragraph, wherein the ADC is an anti-CD25 ADC.

21. A composition, method, use, or kit according to paragraph 20, wherein the anti-CD25 ADC is ADCX25 or ADCT-301.

22. A composition, method, use, or kit according to any previous paragraph, wherein the disorder is a proliferative disease.

23. The composition, method, use, or kit of paragraph 22, wherein the disorder is cancer.

24. The composition, method, use, or kit according any previous paragraph, wherein the individual has, or has been has been determined to have, a disorder characterised by the presence of a neoplasm comprising both CD25+ve and CD25−ve cells.

25. The composition, method, use, or kit according any previous paragraph, wherein the individual has, or has been has been determined to have, a disorder characterised by the presence of a neoplasm comprising, or composed of, CD25−ve neoplastic cells.

26. The composition, method, use, or kit according to either of paragraphs 24 or 25, wherein the neoplasm is all or part of a solid tumour.

27. The composition, method, use, or kit of any previous paragraph, wherein the disorder is selected from the group comprising:
Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL);
leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL);
pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

1a. A method for treating a disorder in an individual, the method comprising administering to the individual an effective amount of an ADC and 5-fluorouracil.

2a. A first composition comprising an ADC for use in a method of treating a disorder in an individual, wherein the treatment comprises administration of the first composition in combination with a second composition comprising 5-fluorouracil.

3a. A first composition comprising 5-fluorouracil for use in a method of treating a disorder in an individual, wherein the treatment comprises administration of the first composition in combination with a second composition comprising an ADC.

4a. Use of an ADC in the manufacture of a medicament for treating a disorder in an individual, wherein the medicament comprises an ADC, and wherein the treatment comprises administration of the medicament in combination with a composition comprising 5-fluorouracil.

5a. Use of 5-fluorouracil in the manufacture of a medicament for treating a disorder in an individual, wherein the medicament comprises 5-fluorouracil, and wherein the treatment comprises administration of the medicament in combination with a composition comprising an ADC.

6a. A kit comprising:
a first medicament comprising an ADC;
a second medicament comprising 5-fluorouracil; and, optionally,
a package insert comprising instructions for administration of the first medicament to an individual in combination with the second medicament for the treatment of a disorder.

7a. A kit comprising a medicament comprising an ADC and a package insert comprising instructions for administration of the medicament to an individual in combination with a composition comprising 5-fluorouracil for the treatment of a disorder.

8a. A kit comprising a medicament comprising 5-fluorouracil and a package insert comprising instructions for administration of the medicament to an individual in combination with a composition comprising an ADC for the treatment of a disorder.

9a. A pharmaceutical composition comprising an ADC and 5-fluorouracil.

10a. A method of treating a disorder in an individual, the method comprising administering to the individual an effective amount of the composition of paragraph 9.

11a. The composition of paragraph 9 for use in a method of treating a disorder in an individual.

12a. The use of the composition of paragraph 9a in the manufacture of a medicament for treating a disorder in an individual.

13a. A kit comprising the composition of paragraph 9a and a set of instructions for administration of the medicament to an individual for the treatment of a disorder.

14a. The composition, method, use, or kit according to any previous paragraph, wherein the treatment comprises administering the ADC before 5-fluorouracil, simultaneous with 5-fluorouracil, or after 5-fluorouracil.

15a. The composition, method, use, or kit according to any previous paragraph, wherein the treatment further comprises administering a chemotherapeutic agent.

16a. The composition, method, use, or kit according to any previous paragraph, wherein the individual is human.

17a. The composition, method, use, or kit according to any preceding paragraph, wherein the individual has a disorder or has been determined to have a disorder.

18a. The composition, method, use, or kit according to paragraph 17a, wherein the individual has, or has been has been determined to have, a cancer which expresses CD25 or CD25+ tumour-associated non-tumour cells, such as CD25+ infiltrating T-cells.

19a. The composition, method, use, or kit according to any one of the preceding paragraphs, wherein the treatment:
a) effectively treats a broader range of disorders,
b) effectively treats resistant, refractory, or relapsed disorders,
c) has an increased response rate, and/or
d) has increased durability;
as compared to treatment with either the ADC or 5-fluorouracil alone.

20a. A composition, method, use, or kit according to any previous paragraph, wherein the ADC is an anti-CD25 ADC.

21a. A composition, method, use, or kit according to paragraph 20a, wherein the anti-CD25 ADC is ADCX25 or ADCT-301.

22a. A composition, method, use, or kit according to any previous paragraph, wherein the disorder is a proliferative disease.

23a. The composition, method, use, or kit of paragraph 22a, wherein the disorder is cancer.

24a. The composition, method, use, or kit according any previous paragraph, wherein the individual has, or has been has been determined to have, a disorder characterised by the presence of a neoplasm comprising both CD25+ve and CD25−ve cells.

25a. The composition, method, use, or kit according any previous paragraph, wherein the individual has, or has been has been determined to have, a disorder characterised by the presence of a neoplasm comprising, or composed of, CD25−ve neoplastic cells.

26a. The composition, method, use, or kit according to either of paragraphs 24a or 25a, wherein the neoplasm is all or part of a solid tumour.

27a. The composition, method, use, or kit of any previous paragraph, wherein the disorder is selected from the group comprising:
Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL);
leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL);
pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

### EXAMPLES

In the following examples:
the FTP is preferably CD25.
Cell lines expressing CD25 suitable for use in the examples include L540, Karpas299, Sudhl1, HDLM-2 cells.
Disease A—Diffuse Large B-cell Lymphoma/DLBC is an aggressive type of non-Hodgkin lymphoma that develops from the B-cells in the lymphatic system. It constitutes the largest subgroup of non-Hodgkin lymphoma.
Disease B—Mantle Cell Lymphoma/MCL is a rare B-cell NHL that most often affects men over the age of 60. The disease may be aggressive (fast growing) but it can also behave in a more indolent (slow growing) fashion in some patients. MCL comprises about five percent of all NHLs.
Disease C—Follicular lymphoma/FL is a fairly indolent type of NHL with long survival time but for which it is very difficult to achieve a cure; it can also transform into more aggressive forms of lymphoma.

### Example 1

To show that a PBD-ADC can induce ICD and therefore can be a suitable combination agent with immune-oncology (10) drugs, cell lines expressing a first target protein (FTP), will be incubated for 0, 6, 24 and 48 hours with etoposide (negative control) and oxaliplatin (positive control), 1 μg/mL ADC, 1 μg/mL anti-FTP (the antibody in ADC) and 1 μg/mL of B12-SG3249 (a non-binding control ADC with the same PBD payload as ADC).

After Incubation, the amount of AnnexinV−/PI+ (early apoptotic cells) will be measured by Flow cytometry together with the upregulation of surface calreticulin and HSP-70. ER stress will be measured by Northern blot analyses of IRE1 phosphorylation, ATF4 and JNK phosphorylation.

### Example 2

In a separate experiment, cell lines expressing FTPs will be incubated for 0, 6, 24 and 48 hours with etoposide (negative control) and oxaliplatin (positive control), 1 μg/mL ADC (ADC targeting FTP with a PBD dimer warhead), 1 μg/mL anti-FTP (the antibody in ADC) and 1 μg/mL of B12-SG3249 (a non-binding control ADC with the same PBD payload as ADC).

After incubation, the cells are washed, and fed to human Dendritic cells (DCs) for an additional 24 h. Activation of the DCs is subsequently measured by increased surface expression of CD86 on the DC population (as determined by Flow cytometry) and by measuring DC mediated release of IL-8 and MIP2.

### Example 3

The purpose of this study is to preliminarily assess the safety, tolerability, pharmacological and clinical activity of this combination

The following cancer types have been chosen for

study: Disease A, Disease B, and Disease C

Evidence for efficacy as single agents exists for both drugs:
ADC (see, for example, WO2014/057119, WO2016/083468, and WO2016/166341)
a Gemcitabine or 5-fluorouracil (see K S Peggs et al. 2009, Clinical and Experimental Immunology, 157: 9-19 [doi:10.1111/j.1365-2249.2009.03912.x])

This primary purpose of this study is to explore whether these agents can be safely combined, and if so, will identify the dose(s) and regimens appropriate for further study. The study will also assess whether each combination induces pharmacologic changes in tumor that would suggest potential clinical benefit.

In addition, it will provide preliminary evidence that a combination may increase the response rate and durability of response compared with published data for treatment with single agent ADC or Gemcitabine or 5-fluorouracil.

Each disease group may include a subset of patients previously treated with Gemcitabine or 5-fluorouracil to explore whether combination therapy might overcome resistance to Gemcitabine or 5-fluorouracil therapy. For each disease, it is not intended to apply specific molecular selection as the data available at present generally do not support excluding patients on the basis of approved molecular diagnostic tests.

Rationale for ADC Starting Dose

The RDE for already established for ADC (in ug/kg administered every three weeks) will be used for all patients in this study. To ensure patient safety, a starting dose below the RDE will be used; the starting dose level will be one where patient benefit could still be demonstrated in study ADC1, suggesting that patients enrolled at such dose level will gain at least some benefit by taking part.

Rationale for Gemcitabine or 5-Fluorouracil Starting Dose

The RDE for already established for Gemcitabine or 5-fluorouracil (in ug/kg administered every three weeks) will be used for all patients in this study. To ensure patient safety, a starting dose below the RDE will be used; the starting dose level will be one where patient benefit could still be demonstrated in study SA1, suggesting that patients enrolled at such dose level will gain at least some benefit by taking part.

Objectives and Related Endpoints

| Objective | Endpoint |
|---|---|
| Primary Objective | |
| To characterize the safety and tolerability | Frequency and severity of treatment- |
| of ADC in combination with Gemcitabine or | emergent AEs and SAEs |
| 5-fluorouracil, and to identify the | Changes between baseline and post- |
| recommended dose and schedules for | baseline laboratory parameters and vital |
| future studies | signs |
| | Incidence of dose limiting toxicities (DLTs) |
| | during the first cycle of treatment (dose |
| | escalation only) |
| | Frequency of dose interruptions and dose |
| | reductions |
| Secondary Objectives | |
| To evaluate the clinical activity of the | ORR, DOR, PFS, OS |
| combination of ADC with Gemcitabine or 5- | AUC and Cmax for each compound |
| fluorouracil | Anti-Drug-Antibodies (ADAs) before, during |
| To characterize the pharmacokinetic (PK) | and after treatment with ADC |
| profile of each of the two compounds ADC | |
| and Gemcitabine or 5-fluorouracil | |
| Evidence for immunogenicity and ADAs to | |
| ADC | |
| Exploratory Objectives | |
| To examine potential correlation of PK | Correlation coefficients between AUC |
| profiles with safety/tolerability and efficacy | and/or Cmax of each compound or a |
| To characterize changes in the immune | compound measure and any of the safety |
| infiltrate in tumors | or efficacy variables |
| To characterize changes in circulating | Immunohistochemistry of pre- and on- |
| levels of cytokines in plasma and markers | treatment tumor biopsies, |
| of activation in circulating immune cells | Measurements (e.g. via ELISA) of |
| | immunologically relevant cytokines in |
| | plasma or serum; staining levels for |
| | activation markers of circulating immune |
| | cells (e.g. FACS) |

Study Design

This phase Ib, multi-center, open-label study to characterize the safety, tolerability, pharmacokinetics (PK), pharmacodynamics (PD) and antitumor activity of the ADC in combination with Gemcitabine or 5-fluorouracil, in patients with disease A, disease B, and disease C.

The study is comprised of a dose escalation part followed by a dose expansion part.

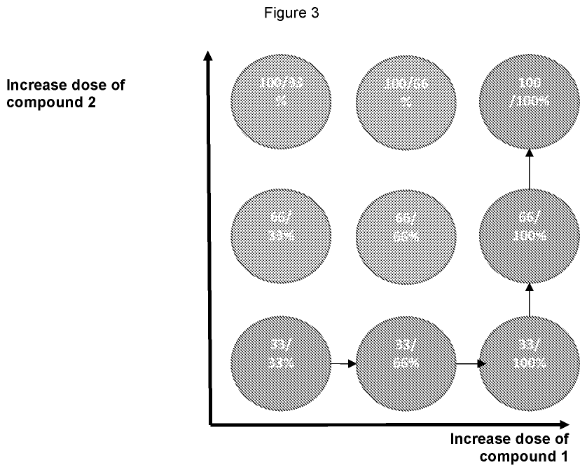

Dose escalation will start with reduced starting doses (compared to their respective recommended phase 2 or licensed dose levels), for both ADC and Gemcitabine or 5-fluorouracil, to guarantee patient safety. Starting doses will be 33% (or 50%) of the RDE for each compound. Subsequently, doses will be first escalated for Gemcitabine or 5-fluorouracil until the RDE or licensed dose has been reached, or a lower dose if necessary for tolerability reasons. Then, the dose for ADC will be escalated, until the RDE for combination treatment is reached. This is visualized in FIG. 3.

If the dose combination is determined to be safe, it may be tested in additional patients to confirm the safety and tolerability at that dose level. Further tailoring of the dose of each compound may be conducted, and/or the regimen may be modified.

The dose escalation of the combination will be guided by a Bayesian Logistic Regression Model (BLRM) based on any Dose Limiting Toxicities (DLTs) observed in the first (or first two, TBC) cycles of therapy. Use of a BLRM is a well-established method to estimate the maximum tolerated dose (MTD)/recommended dose for expansion (RDE) in cancer patients. The adaptive BLRM will be guided by the Escalation With Overdose Control (EWOC) principle to control the risk of DLT in future patients on the study. The use of Bayesian response adaptive models for small datasets has been accepted by FDA and EMEA (“Guideline on clinical trials in small populations”, Feb. 1, 2007) and endorsed by numerous publications (Babb et al. 1998, Neuenschwander et al. 2008).

The decisions on new dose combinations are made by the Investigators and sponsor study personnel in a dose escalation safety call (DESC) based upon the review of patient tolerability and safety information (including the BLRM summaries of DLT risk, if applicable) along with PK, PD and preliminary activity information available at the time of the decision.

Once the MTD(s)/RDE is determined for the combination, the expansion part of the study may be initiated to further assess the safety, tolerability and preliminary efficacy.
For combinations with 10, changes in the immune infiltrate in tumors will also be characterized following combination treatment in the target disease indications.

Given the available prior clinical experience with the agents in this study, it is expected that in most cases a combination dose can be identified without testing a large number of dose levels or schedules. To assess the pharmacodynamic activity of the combinations, patients will be asked to undergo a tumor biopsy at baseline and again after approximately two cycles of therapy.
For 10 combo: The extent of the change in tumor infiltration by immune cells including lymphocytes and macrophages will contribute to a decision on any potential benefit,

Dose Escalation Part

During the dose escalation part of the study, patients will be treated with a fixed dose of ADC administered iv., and increasing doses of Gemcitabine or 5-fluorouracil until the RDE for Gemcitabine or 5-fluorouracil has been reached. Subsequently, doses of ADC are increased (in different cohorts) while the dose for Gemcitabine or 5-fluorouracil is kept constant.

Two to approximately 3 or 4 patients with disease A, disease B or disease C will be treated in each escalation cohort until the determination of MTD(s)/RDE(s) is determined.

There will be a 24-hour observation before enrolling the second patient at Dose Level 1. The DLT observation period at each dose level is either 1 cycle (3 weeks) or 2 cycles (6 weeks) as mandated by the appropriate authorities for 10 therapies, after which it will be determined whether to escalate to the next dose level, stay at the current dose level, or de-escalate to the previous dose level for the next cohort. There will be no de-escalation from Dose Level 1. Intrapatient dose escalation is not permitted.

Dose escalation is not permitted unless 2 or more patients have complete DLT information through the first cycle in any given dose level. Dose escalation will be determined by using a mCRM with a target DLT rate of 30% and an equivalence interval of 20% to 35%, and with dose escalation-with-overdose-control (EWOC) and no dose skipping.

Patients will be assigned to a cohort that is actively enrolling. Dose escalation will be performed in each combination following the completion of one cycle of treatment.

Safety assessments including adverse events (AEs) and laboratory values will be closely monitored for all enrolled patients in order to identify any DLTs. A single MTD/RDE will be defined; a disease-specific MTD/RDE will not be established.

The mCRM will be implemented for DE under the oversight of a Dose Escalation Steering Committee (DESC). The DESC will confirm each escalating dose level after reviewing all available safety data. PK data from patients in that dose level and prior dose levels may also inform decision making. The DESC may halt dose escalation prior to determining the MTD based on emerging PK, PD, toxicity or response data.

Additional patients may be included at any dose level to further assess the safety and tolerability if at least 1 patient in the study has achieved a partial response or better, or if further evaluation of PK or PD data is deemed necessary by the DESC to determine the RDE.

Dose Escalation will be stopped after 3 cohorts (or at least 6 patients) are consecutively assigned to the same dose level. If the MTD is not reached, the recommended dose for expansion (RDE) will be determined. Prior to the determination of the MTD/RDE a minimum of 6 patients must have been treated with the combination.

It is intended that paired tumor biopsies will be obtained from patients during dose escalation. Analysis of these biopsies will contribute to a better understanding of the relationship between the dose and the pharmacodynamic activity of the combination.

Safety Oversight by the Dose Escalation Steering Committee

A DESC comprised of ADC Therapeutics and the investigators will review patient safety on an ongoing basis during the DE to determine if the dose escalation schedule prescribed by the mCRM warrants modification. In addition to safety observations, PK and/or PD data may also inform decision making. Intermediate doses may be assigned after agreement between ADC Therapeutics and investigators. The DESC may continue to provide oversight during Part 2. No formal Data Safety Monitoring Board (DSMB) will be used.

Dose Expansion Part

Once the MTD/RDE has been declared, dose expansion part may begin. The main objective of the expansion part is to further assess the safety and tolerability of the study treatment at the MTD/RDE and to gain a preliminary understanding of the efficacy of the combination compared to historical single agent efficacy data.

An important exploratory objective is to assess changes in the immune infiltrate in tumor in response to treatment. This will be assessed in paired tumor biopsies collected from patients, with a minimum of ten evaluable biopsy pairs (biopsy specimens must contain sufficient tumor for analysis) in patients treated at the MTD/RDE. If this is not feasible, collection of these biopsies may be stopped. A minimum of 10 to 20 patients are planned to be treated in each investigational arm,

Several different investigational arms will open, one per disease. A total of nine investigational arms may be run in the dose expansion. Should enrollment for any of these groups not be feasible, then enrollment to that group may be closed before the 10 to 20 patients target is met.

In each treatment group a maximum of approximately six patients who have received and progressed on prior single administration (i.e. not in combination) Gemcitabine or 5-fluorouracil therapy will be allowed to be treated. This number may be increased if a combination shows promise of overcoming resistance to prior treatment with single administration Gemcitabine or 5-fluorouracil.

Patient Population

The study will be conducted in adult patients with advanced Disease A, Disease B or Disease C as outlined above. The investigator or designee must ensure that only patients who meet all the following inclusion and none of the exclusion criteria are offered treatment in the study.

Inclusion criteria
Patients eligible for inclusion in this study have to meet all of the following criteria:
1. Written informed consent must be obtained prior to any procedures
2. Age 18 years.
3. Patients with advanced/metastatic cancer, with measurable disease as determined by RECIST version 1.1, who have progressed despite standard therapy or are intolerant to standard therapy, or for whom no standard therapy exists. Patients must fit into one of the following groups:
   Disease A
   Disease B
   Disease C
4. ECOG Performance Status 0-1 (or 2 TBC)
5. TBC: Patient must have a site of disease amenable to biopsy, and be a candidate for tumor biopsy according to the treating institution's guidelines. Patient must be willing to undergo a new tumor biopsy at baseline, and again during therapy on this study.
6. Prior therapy with Gemcitabine or 5-fluorouracil or related compounds (i.e. same MOA) is allowed

Exclusion Criteria

Patients eligible for this study must not meet any of the following criteria:
1. History of severe hypersensitivity reactions to other mAbs (OR to same backbone mAb as in ADC OR to same 10 mAb if applicable)
2. Known history of positive serum human ADA to backbone of mAb as in ADC
3. Central Nervous System (CNS) disease only (if applicable)
4. Symptomatic CNS metastases or evidence of leptomeningeal disease (brain MRI or previously documented cerebrospinal fluid (CSF) cytology)
   Previously treated asymptomatic CNS metastases are permitted provided that the last treatment (systemic anticancer therapy and-or local radiotherapy) was completed >=8 weeks prior to 1^{st} day of dosing, except usage of low dose steroids on a taper is allowed)
   Patients with discrete dural metastases are eligible.
5. Patient having out of range laboratory values defined as:
   Serum creatinine <=1.5×ULN. If serum creatinine >1.5, the creatinine clearance (calculated using Cockcroft-Gault formula, or measured) must be >60 mL/min/1.73m2 for a patient to be eligible
   Total bilirubin >1.5×ULN, except for patients with Gilbert's syndrome who are excluded if total bilirubin >3.0×ULN or direct bilirubin >1.5×ULN
   Alanine aminotransferase (ALT) >3×ULN, except for patients that have tumor involvement of the liver, who are excluded if ALT>5×ULN
   Aspartate aminotransferase (AST)>3×ULN, except for patients that have tumor involvement of the liver, who are excluded if AST>5×ULN
   Absolute neutrophil count<1.0×10e9/L
   Platelet count<75×10e9/L
   Hemoglobin (Hgb)<8 g/dL
   Potassium, magnesium, calcium or phosphate abnormality>CTCAE grade 1 despite appropriate replacement therapy
6. Impaired cardiac function or clinically significant cardiac disease, including any of the following:
   Clinically significant and/or uncontrolled heart disease such as congestive heart failure requiring treatment (NYHA grade III or IV) or uncontrolled hypertension defined by a Systolic Blood Pressure (SBP) 160 mm Hg and/or Diastolic Blood Pressure (DBP) 100 mm Hg, with or without anti-hypertensive medication.
   QTcF >470 msec for females or >450 msec for males on screening ECG using Fridericia's correction, congenital long QT syndrome
   Acute myocardial infarction or unstable angina pectoris <3 months (months prior to study entry
   Clinically significant valvualr disease with documented compromise in cardiac function
   Symptomatic pericarditis
   History of or ongoing documented cardiomyopathy
   Left Ventricular Ejection Fraction (LVEF)<40%, as determined by echocardiogram (ECHO) or Multi gated acquisition (MUGA) scan
   History or presence of any clinically significant cardiac arrhythmias, e.g. ventricular, supraventricular, nodal arrhythmias, or conduction abnormality (TBC qualifier: . . . requiring a pacemaker or not controlled with medication)
   Presence of unstable atrial fibrillation (ventricular response rate>100 bpm).
      NOTE: Patients with stable atrial fibrillation can be enrolled provided they do not meet other cardiac exclusion criteria.
   Complete left bundle branch block (LBBB), bifascicular block
   Any clinically significant ST segment and/or T-wave abnormalities
7. Toxicity attributed to prior 10 therapy that led to discontinuation of therapy. Adequately treated patients for drug-related skin rash or with replacement therapy for endocrinopathies are not excluded, provided these toxicities did not lead to the discontinuation of prior treatment.
8. Patients with active, known or suspected autoimmune disease. Subjects with vitiligo, type I diabetes mellitus, residual hypothyroidism due to autoimmune condition only requiring hormone replacement, psoriasis not requiring systemic treatment, or conditions not expected to recur in the absence of an external trigger are permitted to enroll, provided the trigger can be avoided.
9. Human Immunodeficiency Virus (HIV), or active Hepatitis B (HBV) or Hepatitis C (HCV) virus infection
   Testing is not mandatory to be eligible. Testing for HCV should be considered if the patient is at risk for having undiagnosed HCV (e.g. history of injection drug use).
10. Malignant disease, other than that being treated in this study. Exceptions to this exclusion include the following: malignancies that were treated curatively and have not recurred within 2 years prior to study treatment; completely resected basal cell and squamous cell skin cancers; any malignancy considered to be indolent and that has never required therapy; and completely resected carcinoma in situ of any type.
11. Systemic anti-cancer therapy within 2 weeks of the first dose of study treatment. For cytotoxic agents that have major delayed toxicity, e.g. mitomycin C and nitrosoureas, 4 weeks is indicated as washout period. For patients receiving anticancer immunotherapies such as CTLA-4 antagonists, 6 weeks is indicated as the washout period.
12. Active diarrhea CTCAE grade 2 or a medical condition associated with chronic diarrhea (such as irritable bowel syndrome, inflammatory bowel disease)
13. Presence of 2: CTCAE grade 2 toxicity (except alopecia, peripheral neuropathy and ototoxicity, which are excluded if >=CTCAE grade 3) due to prior cancer therapy.
14. Active infection requiring systemic antibiotic therapy.
15. Active ulceration of the upper GI tract or GI bleeding
16. Active bleeding diathesis or on oral anti-vitamin K medication (except low-dose warfarin and aspirin or equivalent, as long as the INR<=2.0)
17. Active autoimmune disease, motor neuropathy considered of autoimmune origin, and other CNS autoimmune disease
18. Patients requiring concomitant immunosuppressive agents or chronic treatment with corticoids except:
   replacement dose steroids in the setting of adrenal insufficiency
   topical, inhaled, nasal and ophthalmic steroids are allowed
19. Use of any live vaccines against infectious diseases (e.g. influenza, varicella, pneumococcus) within 4 weeks of initiation of study treatment (NB the use of live vaccines is not allowed through the whole duration of the study)
20. Use of hematopoietic colony-stimulating growth factors (e.g. G-CSF, GMCSF, M-CSF)<2 weeks prior start of study drug. An erythroid stimulating agent is allowed as long as it was initiated at least 2 weeks prior to the first dose of study treatment.
21. Major surgery within 2 weeks of the first dose of study treatment (NB mediastinoscopy, insertion of a central venous access device, or insertion of a feeding tube are not considered major surgery).
22. Radiotherapy within 2 weeks of the first dose of study drug, except for palliative radiotherapy to a limited field, such as for the treatment of bone pain or a focally painful tun1or mass. To allow for assessment of response to treatment, patients must have remaining measurable disease that has not been irradiated
23. Participation in an interventional, investigational study within 2 weeks of the first dose of study treatment.
24. Any medical condition that would, in the investigator's judgment, prevent the patient's participation in the clinical study due to safety concerns, compliance with clinical study procedures or interpretation of study results.
25. Sexually active males unless they use a condom during intercourse while taking drug and for 90 days after stopping study treatment and should not father a child in this period. A condom is required to be used also by vasectomized men in order to prevent delivery of the drug via seminal fluid.
26. Pregnant or lactating women, where pregnancy is defined as the state of a female after conception and until the termination of gestation, confirmed by a positive hCG laboratory test. In rare cases of an endocrine-secreting tumor, hCG levels may be above normal limits but with no pregnancy in the patient. In these cases, there should be a repeat serum hCG test (with a non-rising result) and a vaginal/pelvic ultrasound to rule out pregnancy. Upon confirmation of results and discussion with the Medical representative, these patients may enter the study.
27. Women of child-bearing potential, defined as all women physiologically capable of becoming pregnant, unless they are using highly effective methods of contraception during study treatment and for 90 days after the last any dose of study treatment. Highly effective contraception methods include:
   Total abstinence (when this is in line with the preferred and usual lifestyle of the patient. Periodic abstinence (e.g., calendar, ovulation, symptothermal, post-ovulation methods) and withdrawal are not acceptable methods of contraception
   Female sterilization (have had surgical bilateral oophorectomy with or without hysterectomy), total hysterectomy or tubal ligation at least 6 weeks before taking study treatment. In case of oophorectomy alone, only when the reproductive status of the woman has been confirmed by follow up hormone level assessment
   Male sterilization (at least 6 months prior to screening). For female patients on the study the vasectomized male partner should be the sole partner for that patient.
   Use of oral (estrogen and progesterone), injected or implanted combined hormonal methods of contraception or placement of an intrauterine device (IUD) or intrauterine system (IUS) or other forms of hormonal contraception that have comparable efficacy (failure rate <1%), for example hormone vaginal ring or transdermal hormone contraception.
      In case of use of oral contraception, women should have been stable on the same pill for a minimum of 3 months before taking study treatment.
      Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy) or tubal ligation at least 6 weeks ago. In the case of oophorectomy alone, only when the reproductive status of the woman has been confirmed by follow up hormone level assessment is she considered not of child bearing potential.

Dose-Limiting Toxicities and Dose Modification Guidelines

A dose-limiting toxicity (DLT) is defined as any of the following events thought to be at least possibly related to ADC per investigator judgment that occurs during the 21-day DLT evaluation period. Toxicity that is clearly and directly related to the primary disease or to another etiology is excluded from this definition.

DLT Definitions

A hematologic DLT is defined as:
Grade 3 or 4 febrile neutropenia or neutropenic infection
Grade 4 neutropenia lasting >7 days
Grade 4 thrombocytopenia
Grade 3 thrombocytopenia with clinically significant bleeding, or Grade 3 thrombocytopenia requiring a platelet transfusion
Grade 3 anemia that requires transfusion
Grade 4 anemia

A non-hematologic DLT is defined as:
Grade 4 non-hematologic toxicity
Grade 3 non-hematologic toxicity lasting >3 days despite optimal supportive care or medical intervention
A case of Hy's law (AST and/or ALT>3×ULN and bilirubin >2×ULN, and without initial findings of cholestasis (serum alkaline phosphatase (ALP) activity <2×ULN) and no other reason that could explain the combination of increased transaminases and serum total bilirubin, such as viral hepatitis A, B, or C, preexisting or acute liver disease, or another drug capable of causing the observed injury)
Grade 3 or higher hypersensitivity/infusion-related reaction (regardless of premedication). A grade 3 hypersensitivity/infusion-related reaction that resolves within 8 hours after onset with appropriate clinical management does not qualify as a DLT.
LVEF decrease to <40% or >20% decrease from baseline
Grade 4 tumor lysis syndrome (Grade 3 TLS will not constitute DLT unless it leads to irreversible end-organ damage)

The following conditions are not considered non-hematologic DLT:
Grade 3 fatigue for s 7 days
Grade 3 diarrhea, nausea, or vomiting in the absence of premedication that responds to therapy and improves by at least 1 grade within 3 days for Grade 3 events or to s Grade 1 within 7 days.
AST or ALT elevation >5×ULN but s 8×ULN, without concurrent elevation in bilirubin, that downgrades to s Grade 2 within 5 days after onset.
Grade 3 serum lipase or serum amylase for s 7 days if without clinical signs or symptoms of pancreatitis

Patients who experience a DLT that resolves or stabilizes with appropriate medical management may continue treatment at the discretion of the investigator in consultation with the sponsor.

Dose Modifications

Guidelines for management of specific toxicities are detailed in the table below. For management of events not specified in the tables, the following may serve as a guidance to investigators:

| AE Grade | ADC Management Guideline |
|---|---|
| 1 | No dose adjustment is required. |
| 2 | First occurrence: |
| | Consider holding one or both drugs until improvement to ≤Grade 1 or |
| | baseline. Up to 1 dose of one or both drugs may be skipped to permit |
| | improvement. If improvement to ≤Grade 1 or baseline occurs within 21 |
| | days from the last scheduled (but missed) dose of one or both drugs, |
| | continue one or both drugs at the original assigned dose level in subsequent |
| | treatment cycles. |
| | If improvement to ≤Grade 1 or baseline does not occur within 21 days from |
| | the last scheduled (but missed) dose, permanently discontinue one or both |
| | drugs. |
| | Second occurrence: |
| | Hold one or both drugs until improvement to ≤Grade 1 or baseline. Up to 1 |
| | dose of one or both drugs may be skipped to permit resolution. If |
| | improvement to ≤Grade 1 or baseline occurs within 21 days from the last |
| | scheduled (but missed) dose, continue one or both drugs at 1 dose level |
| | below the original assigned dose in subsequent treatment cycles. If |
| | improvement to ≤Grade 1 or baseline does not occur within 21 days from |
| | the last scheduled (but missed) dose, permanently discontinue one or both |
| | drugs. |
| | Third occurrence: |
| | Permanently discontinue one or both drugs. |
| 3 | First occurrence: |
| | Hold one or both drugs until improvement to ≤Grade 1 or baseline. Up to 1 |
| | dose of one or both drugs may be skipped to permit improvement, then |
| | continue at 1 dose level below the original assigned dose in subsequent |
| | treatment cycles. |
| | Second occurrence: |
| | Permanently discontinue one or both drugs |
| 4 | Permanently discontinue one or both drugs. |

### Example 4

In vivo efficacy study testing combination of sur301 with gemcitabine in the CT26 syngeneic modelFemale BALB/c mice (BALB/cNCrI, Charles River) were ten weeks old on Day 1 of the study and had a body weight (BW) range of 17.2 to 22.0 g.

On the day of implant, cultured CT26 cells were harvested during log phase growth and resuspended in PBS at a concentration of 3×10⁶ cells/mL. Tumors were initiated by subcutaneously implanting 3×10⁵ CT26 cells (0.1 mL suspension) into the right flank of each test animal. Tumors were monitored as their volumes approached the target range of 80-120 mm³. Tumor were measured in two dimensions using calipers, and volume was calculated using the formula:

Tumor Volume (mm³)=w2×l/2

where w=width and l=length, in mm, of the tumor. Tumor weight may be estimated with the assumption that 1 mg is equivalent to 1 mm3 of tumor volume.

Fifteen days after tumor cell implantation, on Day 1 of the study, animals were sorted into groups (n=10/group) with individual tumor volumes of 75 to 144 mm³, and group mean tumor volumes of 104-106 mm³.

All doses were administered intraperitoneally (i.p.). The dosing volume was 0.2 mL per 20 grams of body weight (10 mL/kg), and was scaled to the body weight of each individual animal.

Gemcitabine was administered at 80 mg/kg q3d×4 which corresponded to Days 1, 4, 7 and 10.

The antibody component of ADC×25 specifically binds human CD25. Accordingly, ADC×25 cannot be used in murine in vivo studies where mice express their native murine CD25. Accordingly, an equivalent ADC that can be used in murine in vivo studies was created. In Arce Vargas et al., 2017, Immunity 46, 1-10, Apr. 18, 2017 (http://dx.doi.org/10.1016/j.ummuni.2017.03.013) a Fc enhanced version of PC61, a rat antibody directed against mouse CD25 was described. The wild-type PC61 was conjugated to the PBD dimer drug-linker SG3249 (the PBD drug-linker used in ADC×25/ADCT-301/Camidanlumab Tesirine) and designated as Surrogate-ADC×25 (also termed SurADC×25 or sADC×25). sADC×25 was administered in monotherapy as single dose on day 1 of the study. In the combination groups with gemcitabine, sADC×25 was administered on Day 5.

Tumors were measured using calipers twice per week, and each animal was euthanized when its tumor reached the endpoint volume of 2000 mm³ or at the end of the study (Day 56), whichever came first.

The results are shown in FIGS. 2A, 2B, and 20, where each set of axes shows the data from a single group of 10 animals (the trace of each individual animal is shown).

Response Rate

| Response summary | PR | CR | TFS |
|---|---|---|---|
| Vehicle | 0 | 0 | 0 |
| sADCx25, 0.1 mg/kg | 0 | 0 | 0 |
| sADCx25, 0.5 mg/kg | 0 | 1 | 0 |
| sADCx25, 1 mg/kg | 0 | 1 | 1 |
| Gemcitabine | 0 | 0 | 0 |
| sADCx25, 0.1 mg/kg + Gemcitabine | 0 | 0 | 0 |
| sADCx25, 0.5 mg/kg + Gemcitabine | 0 | 2 | 2 |
| sADCx25, 1 mg/kg + Gemcitabine | 0 | 1 | 1 |

Response Table Criteria:
Treatment may cause partial regression (PR) or complete regression (CR) of the tumor in an animal.
In a PR response, the tumor volume is 50% or less of its Day 1 volume for three consecutive measurements during the course of the study, and equal to or greater than 13.5 mm3 for one or more of these three measurements.
In a CR response, the tumor volume is less than 13.5 mm3 for three consecutive measurements during the study.
Any animal with a CR response at the end of the study was additionally classified as a tumor-free survivor (TFS).
Animals were scored only once during the study for a PR or CR event and only as CR if both PR and CR criteria were satisfied.

CDI (COEFFICIENT of Drug Interaction) Calculation

Methodology
The Coefficient of Drug Interaction (CDI) in CT26-e570 were assessed for subadditive, additive, or supra-additive (synergism) properties on Day 18, the last day all evaluable animals remained on study (shown by dotted vertical line on each of the plots in FIG. 2A to 2C.
The CDI was determined according to the equation below:

CDI=AB/A×B
Where,
x=mean tumor volume
AB=×AB/×C
A=×A/×C
B=×B/×C
CDI <1 is supra-additive (synergism); CDI=1 is additive; CDI >1 is sub-additive

Groups
(1) gemcitabine only//sADC25 0.1 mg/kg only//gemcitabine+sADC25 0.1 mg/kg →CDI=0.68
(2) gemcitabine only//sADC25 0.5 mg/kg only//gemcitabine+sADC25 0.5 mg/kg →CDI=1.1
(3) gemcitabine only//sADC25 1 mg/kg only//gemcitabine+sADC25 1 mg/kg →CDI=1.33

Comment

The synergism between sADC×25 and gemcitabine is indicated by the <1 CDI when a low concentration (0.1 mg/kg) of sADC×25 is used.

The relatively high efficacy of sADC×25 as a single agent at the higher concentrations of 0.5 and 1 mg/kg means that, in this particular CDI assay, the synergism is more difficult to detect.

However, the response rate data clearly shows the increased efficacy of combined sADC×25 and gemcitabine administration: as single agents, sADC×25 0.5 mg/kg, sADC×25 1 mg/kg, and gemcitabine resulted in a combined total of 1 tumour-free survivor, whereas sADC×25 0.5 mg/kg and sADC×25 1 mg/kg administered in combination with gemcitabine resulted in a combined total of 3 tumour-free survivors.

1.-25. (canceled)

26. A method for treating cancer in an individual, the method comprising administering to the individual an effective amount of an anti-CD25 ADC and Gemcitabine;
wherein the anti-CD25 ADC is a conjugate of formula L-(D^{L})ₚ, where D^{L} is of formula I or II: 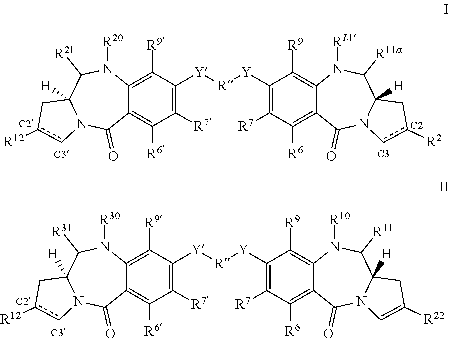
wherein:
L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 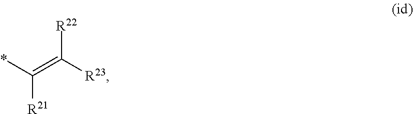
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5; 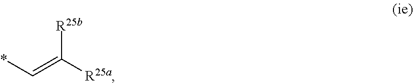
wherein one of R^{25a} and R^{25b} is H and the other is selected from: phenyl, which phenyl is optionally substituted by a group selected from halo, methyl, methoxy; pyridyl; and thiophenyl; and 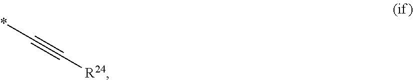
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 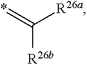
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
[Formula I]
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 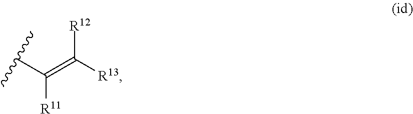
Wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5; 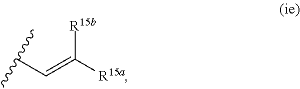
wherein one of R^{15a} and R^{15b} is H and the other is selected from: phenyl, which phenyl is optionally substituted by a group selected from halo, methyl, methoxy; pyridyl; and thiophenyl; and 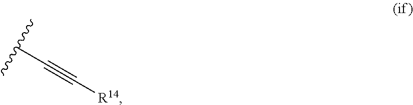
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 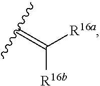
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
[Formula II]
R²² is of formula IIIa, formula IIIb or formula IIIc: 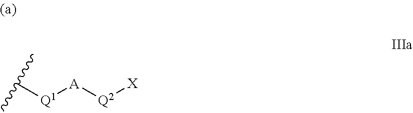
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 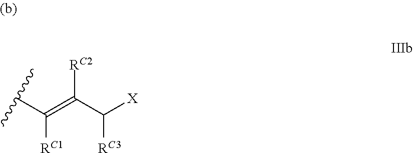
where;
R^{C1}, R^{C2} and R^{C3} are independently selected from H and unsubstituted C₁₋₂ alkyl; 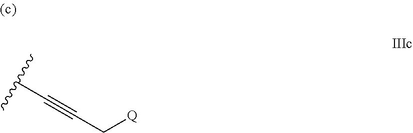
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′} CONHNH—R^{L2′}, 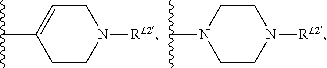
NR^{N}R^{L2′} wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

27. The method according to claim 26, wherein the anti-CD25-ADC has the chemical structure: 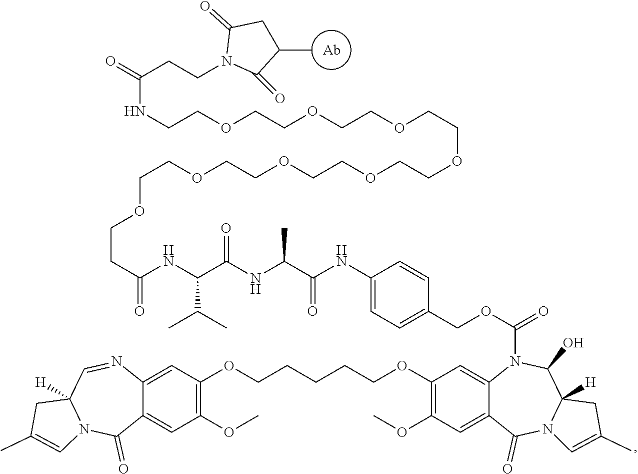
where the Ab is an anti-CD25 antibody, and the DAR is between 1 and 8.

28. The method according to claim 27, wherein the anti-CD25-ADC antibody comprises a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5.

29. The method according to claim 28, wherein the anti-CD25-ADC antibody comprises a VH domain having the sequence according to SEQ ID NO. 1.

30. The method according to claim 28, wherein the anti-CD25-ADC antibody further comprises a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8.

31. The method according to claim 30, wherein the anti-CD25-ADC antibody comprises a VL domain having the sequence according to SEQ ID NO. 2.

32. A pharmaceutical composition comprising an anti-CD25 ADC and Gemcitabine;
wherein the anti-CD25 ADC is a conjugate of formula L-(D^{L})ₚ, where D^{L} is of formula I or II: 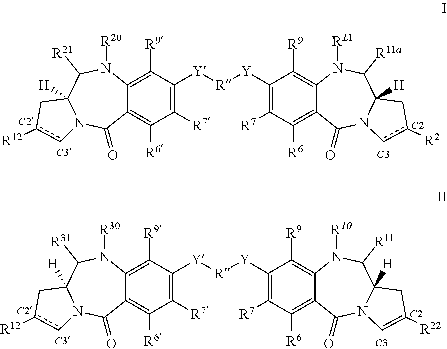
wherein:
L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 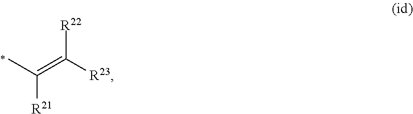
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5; 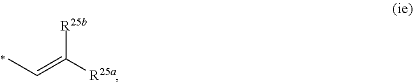
wherein one of R^{25a} and R^{25b} is H and the other is selected from: phenyl, which phenyl is optionally substituted by a group selected from halo, methyl, methoxy; pyridyl; and thiophenyl; and 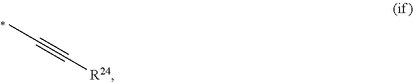
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C₃′,
R¹² is 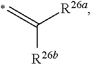
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
[Formula I]
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 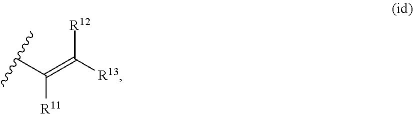
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5; 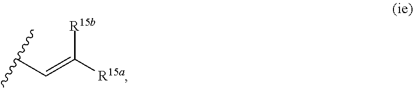
wherein one of R^{15a} and R^{15b} is H and the other is selected from: phenyl, which phenyl is optionally substituted by a group selected from halo, methyl, methoxy; pyridyl; and thiophenyl; and 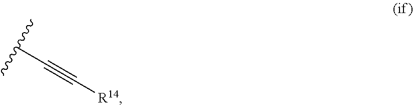
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a singe bond present between C2 and C3,
R² is 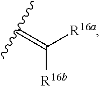
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
[Formula II]
R²² is of formula IIIa, formula IIIb or formula IIIc: 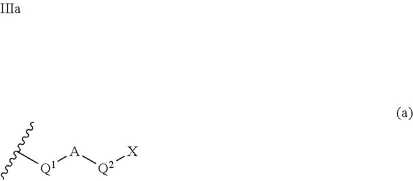
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 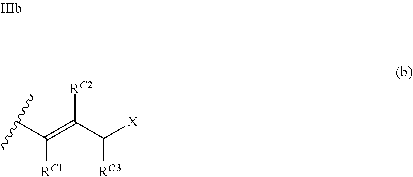
where;
R^{C1}, R^{C2} and R^{C3} are independently selected from H and unsubstituted C₁₋₂ alkyl; 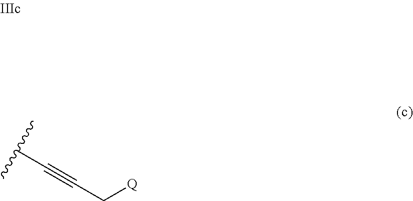
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′}, 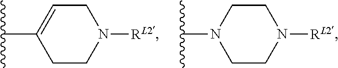
NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

33. A method of treating cancer in an individual, the method comprising administering to the individual an effective amount of the composition of claim 32.

34. The method according to claim 32, wherein the anti-CD25-ADC has the chemical structure: 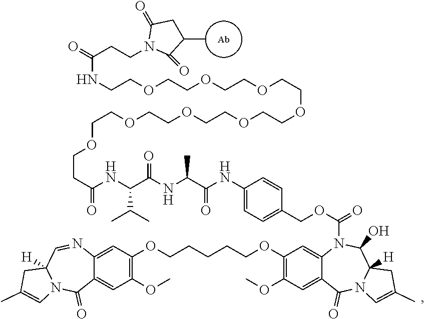
where the Ab is an anti-CD25 antibody, and the DAR is between 1 and 8.

35. The method according to claim 34, wherein the anti-CD25-ADC antibody comprises a VH domain comprising a VH CDR1 with the amino acid sequence of SEQ ID NO. 3, a VH CDR2 with the amino acid sequence of SEQ ID NO. 4, and a VH CDR3 with the amino acid sequence of SEQ ID NO. 5.

36. The method according to claim 35, wherein the anti-CD25-ADC antibody comprises a VH domain having the amino acid sequence according to SEQ ID NO. 1.

37. The method according to claim 35, wherein the anti-CD25-ADC antibody further comprises a VL domain comprising a VL CDR1 with the amino acid sequence of SEQ ID NO. 6, a VL CDR2 with the amino acid sequence of SEQ ID NO. 7, and a VL CDR3 with the amino acid sequence of SEQ ID NO. 8.

38. The method according to claim 37, wherein the anti-CD25-ADC antibody comprises a VL domain having the amino acid sequence according to SEQ ID NO. 2.

39. The method according to claim 26, wherein the treatment further comprises administering a chemotherapeutic agent.

40. The method according to claim 26, wherein the individual is human.

41. The method according to claim 26, wherein the individual has, or has been determined to have, cancer.

42. The method according to claim 26, wherein the individual has, or has been determined to have, a cancer characterised by the presence of a neoplasm comprising both CD25+ve and CD25−ve cells.

43. The method according to claim 26, wherein the individual has, or has been determined to have, a cancer characterised by the presence of a neoplasm comprising, or composed of, CD25−ve neoplastic cells.

44. The method according to claim 26, wherein the cancer or neoplasm is all or part of a solid tumour.

45. The method according to claim 26, wherein the individual has, or has been determined to have, a cancer which expresses CD25 or CD25+ tumour-associated non-tumour cells, such as CD25+ infiltrating T-cells.

46. The method according to claim 26, wherein the cancer is selected from Hodgkin's Lymphoma, non-Hodgkin's Lymphoma, leukemia, pancreatic cancer, breast cancer, colorectal cancer, gastric cancer, oesophageal cancer, lymphoma, melanoma, non-small cell lung cancer, ovarian cancer, hepatocellular carcinoma, renal cell carcinoma, and head and neck cancer.

47. The method according to claim 46, wherein the cancer is a lymphoma selected from diffuse large B-cell lymphoma (DLBCL), follicular lymphoma, (FL), Mantle Cell lymphoma (MCL), chronic lymphatic lymphoma (CLL), and Marginal Zone B-cell lymphoma (MZBL).

48. The method according to claim 46, wherein the cancer is a leukemia selected from Hairy cell leukemia (HCL), Hairy cell leukemia variant (HCL-v), Acute Myeloid Leukaemia (AML), Acute Lymphoblastic Leukaemia (ALL), Philadelphia chromosome-positive ALL (Ph+ALL), and Philadelphia chromosome-negative ALL (Ph−ALL).


SEQUENCE LISTING (8 sequences)

SEQ ID NO: 1 - protein, 127 aa, Artificial Sequence
  AB12 VH
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 2 - protein, 119 aa, Artificial Sequence
  AB12 VL
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 3 - protein, 5 aa, Artificial Sequence
  VH CDR1
RYIIN

SEQ ID NO: 4 - protein, 17 aa, Artificial Sequence
  VH CDR2
RIIPILGVENYAQKFQG

SEQ ID NO: 5 - protein, 6 aa, Artificial Sequence
  VH CDR3
KDWFDY

SEQ ID NO: 6 - protein, 11 aa, Artificial Sequence
  VL CDR1
RASQSVSSYLA

SEQ ID NO: 7 - protein, 7 aa, Artificial Sequence
  VL CDR2
GASSRAT

SEQ ID NO: 8 - protein, 9 aa, Artificial Sequence
  VL CDR3
QQYGSSPLT